SEQUENCE LISTING (104 sequences)

SEQ ID NO: 1 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 2 - protein, 225 aa, Homo sapiens
MAALASSLIRQKREVREPGGSRPVSAQRRVCPRGTKSLCQKQLLILLSKVRLCGGRPARPDRGPEPQLKGIVTKLFCRQGFYLQANPDGSIQGTPEDTSSFTHFNLIPVGLRVVTIQSAKLGHYMAMNAEGLLYSSPHFTAECRFKECVFENYYVLYASALYRQRRSGRAWYLGLDKEGQVMKGNRVKKTKAAAHFLPKLLEVAMYQEPSLHSVPEASPSSPPAP

SEQ ID NO: 3 - protein, 243 aa, Homo sapiens
MAAAIASSLIRQKRQARESNSDRVSASKRRSSPSKDGRSLCERHVLGVFSKVRFCSGRKRPVRRRPEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRKSSGTPTMNGGKVVNQDST

SEQ ID NO: 4 - protein, 245 aa, Homo sapiens
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 5 - protein, 247 aa, Homo sapiens
MAAAIASGLIRQKRQAREQHWDRPSASRRRSSPSKNRGLCNGNLVDIFSKVRIFGLKKRRLRRQDPQLKGIVTRLYCRQGYYLQMHPDGALDGTKDDSTNSTLFNLIPVGLRVVAIQGVKTGLYIAMNGEGYLYPSELFTPECKFKESVFENYYVIYSSMLYRQQESGRAWFLGLNKEGQAMKGNRVKKTKPAAHFLPKPLEVAMYREPSLHDVGETVPKPGVTPSKSTSASAIMNGGKPVNKSKTT

SEQ ID NO: 6 - protein, 207 aa, Homo sapiens
MAEVGGVFASLDWDLHGFSSSLGNVPLADSPGFLNERLGQIEGKLQRGSPTDFAHLKGILRRRQLYCRTGFHLEIFPNGTVHGTRHDHSRFGILEFISLAVGLISIRGVDSGLYLGMNERGELYGSKKLTRECVFREQFEENWYNTYASTLYKHSDSERQYYVALNKDGSPREGYRTKRHQKFTHFLPRPVDPSKLPSMSRDLFHYR

SEQ ID NO: 7 - protein, 216 aa, Homo sapiens
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 8 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 9 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 10 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 11 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 12 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 13 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 14 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 15 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 16 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 17 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 18 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 19 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 20 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATIIQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 21 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 22 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 23 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 24 - protein, 375 aa, Homo sapiens
MKVWLLLGLLLVHEALEDVTGQHLPKNKRPKEPGENRIKPTNKKVKPKIPKMKDRDSANSAPKTQSIMMQVLDKGRFQKPAATLSLLAGQTVELRCKGSRIGWSYPAYLDTFKDSRLSVKQNERYGQLTLVNSTSADTGEFSCWVQLCSGYICRKDEAKTGSTYIFFTEKGELFVPSPSYFDVVYLNPDRQAVVPCRVTVLSAKVTLHREFPAKEIPANGTDIVYDMKRGFVYLQPHSEHQGVVYCRAEAGGRSQISVKYQLLYVAVPSGPPSTTILASSNKVKSGDDISVLCTVLGEPDVEVEFTWIFPGQKDERPVTIQDTWRLIHRGLGHTTRISQSVITVEDFETIDAGYYICTAQNLQGQTTVATTVEFS

SEQ ID NO: 25 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 27 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 28 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 29 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 30 - protein, 1049 aa, Homo sapiens
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 31 - protein, 788 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 32 - protein, 799 aa, Homo sapiens
MPRAPAPLYACLLGLCALLPRLAGLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECLLLHSGKPDNQTCHSLCRDEVITWVDTIVKDDQEAVLCFYKTAKDCVMMFTYVELPSGKSNLTVLREPECGNTPNAMTILLAVVGSILLVGLALLAIWKLLVTIHDRREFAKFQSERSRARYEMASNPLYRKPISTHTVDFTFNKFNKSYNGTVD

SEQ ID NO: 33 - protein, 798 aa, Homo sapiens [sequence omitted]

SEQ ID NO: 34 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 35 - protein, 784 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 36 - protein, 205 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITHSPQAHDNPQEKRLAADDPEVRVLHSIGHSAAPRLFPLAWTVLLLPLLLLQTP

SEQ ID NO: 37 - protein, 213 aa, Homo sapiens
MAPAQRPLLPLLLLLLPLPPPPFARAEDAARANSDRYAVYWNRSNPRFHAGAGDDGGGYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNAVDRPCLRLKVYVRPTNETLYEAPEPIFTSNNSCSSPGGCRLFLSTIPVLWTLLGS

SEQ ID NO: 38 - protein, 238 aa, Homo sapiens
MAAAPLLLLLLLVPVPLLPLLAQGPGGALGNRHAVYWNSSNQHLRREGYTVQVNVNDYLDIYCPHYNSSGVGPGAGPGPGGGAEQYVLYMVSRNGYRTCNASQGFKRWECNRPHAPHSPIKFSEKFQRYSAFSLGYEFHAGHEYYYISTPTHNLHWKCLRMKVFVCCASTSHSGEKPVPTLPQFTMGPNVKINVLEDFEGENPQVPKLEKSISGTSPKREHLPLAVGIAFFLMTFLAS

SEQ ID NO: 39 - protein, 201 aa, Homo sapiens
MRLLPLLRTVLWAAFLGSPLRGGSSLRHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQVSVCCKERKSESAHPVGSPGESGTSGWRGGDTPSPLCLLLLLLLLILRLLRIL

SEQ ID NO: 40 - protein, 228 aa, Homo sapiens
MLHVEMLTLVFLVLWMCVFSQDPGSKAVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 41 - protein, 346 aa, Homo sapiens
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 42 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 43 - protein, 340 aa, Homo sapiens
MGPPHSGPGGVRVGALLLLGVLGLVSGLSLEPVYWNSANKRFQAEGGYVLYPQIGDRLDLLCPRARPPGPHSSPNYEFYKLYLVGGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSLEPGKENLPGDPTSNATSRGAEGPLPPPSMPAVAGAAGGLALLLLGVAGAGGAMCWRRRRAKPSESRHPGPGSFGRGGSLGLGGGGGMGPREAEPGELGIALRGGGAADPPFCPHYEKVSGDYGHPVYIVQDGPPQSPPNIYYKV

SEQ ID NO: 44 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 45 - protein, 402 aa, Homo sapiens [sequence omitted]

SEQ ID NO: 46 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 47 - protein, 155 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEAACVPADDRILCR

SEQ ID NO: 48 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELAPELVISNDKRSFCH

SEQ ID NO: 49 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 50 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 51 - protein, 1388 aa, Homo sapiens
MAPRRNNGQCWCLLMLLSVSTPLPAVTQTRGATETASQGHLDLTQLIGVPLPSSVSFVTGYGGFPAYSFGPGANVGRPARTLIPSTFFRDFAISVVVKPSSTRGGVLFAITDAFQKVIYLGLRLSGVEDGHQRIILYYTEPGSHVSQEAAAFSVPVMTHRWNRFAMIVQGEEVTLLVNCEEHSRIPFQRSSQALAFESSAGIFMGNAGATGLERFTGSLQQLTVHPDPRTPEELCDPEESSASGETSGLQEADGVAEILEAVTYTQASPKEAKVEPINTPPTPSSPFEDMELSGEPVPEGTLETTNMSIIQHSSPKQGSGEILNDTLEGVHSVDGDPITDSGSGAGAFLDIAEEKNLAATAAGLAEVPISTAGEAEASSVPTGGPTLSMSTENPEEGVTPGPDNEERLAATAAGEAEALASMPGEVEASGVAPGELDLSMSAQSLGEEATVGPSSEDSLTTAAAATEVSLSTFEDEEASGVPTDGLAPLTATMAPERAVTSGPGDEEDLAAATTEEPLITAGGEESGSPPPDGPPLPLPTVAPERWITPAQREHVGMKGQAGPKGEKGDAGEELPGPPEPSGPVGPTAGAEAEGSGLGWGSDVGSGSGDLVGSEQLLRGPPGPPGPPGLPGIPGKPGTDVFMGPPGSPGEDGPAGEPGPPGPEGQPGVDGATGLPGMKGEKGARGPNGSVGEKGDPGNRGLPGPPGKKGQAGPPGVMGPPGPPGPPGPPGPGCTMGLGFEDTEGSGSTQLLNEPKLSRPTAAIGLKGEKGDRGPKGERGMDGASIVGPPGPRGPPGHIKVLSNSLINITHGFMNFSDIPELVGPPGPDGLPGLPGFPGPRGPKGDTGLPGFPGLKGEQGEKGEPGAILTEDIPLERLMGKKGEPGMHGAPGPMGPKGPPGHKGEFGLPGRPGRPGLNGLKGTKGDPGVIMQGPPGLPGPPGPPGPPGAVINIKGAIFPIPVRPHCKMPVDTAHPGSPELITFHGVKGEKGSWGLPGSKGEKGDQGAQGPPGPPLDLAYLRHFLNNLKGENGDKGFKGEKGEKGDINGSFLMSGPPGLPGNPGPAGQKGETVVGPQGPPGAPGLPGPPGFGRPGDPGPPGPPGPPGPPAILGAAVALPGPPGPPGQPGLPGSRNLVTAFSNMDDMLQKAHLVIEGTFIYLRDSTEFFIRVRDGWKKLQLGELIPIPADSPPPPALSSNPHQLLPPPNPISSANYEKPALHLAALNMPFSGDIRADFQCFKQARAAGLLSTYRAFLSSHLQDLSTIVRKAERYSLPIVNLKGQVLFNNWDSIFSGHGGQFNMHIPIYSFDGRDIMTDPSWPQKVIWHGSSPHGVRLVDNYCEAWRTADTAVTGLASPLSTGKILDQKAYSCANRLIVLCIENSFMTDARK

SEQ ID NO: 52 - protein, 1754 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTKDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 53 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSREEDSLEAGLPLQVRGYPEEKKEEEGSANRRPEDQELESLSAIEAELEKVAHQLQALRRG

SEQ ID NO: 54 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 55 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 56 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 57 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 58 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 59 - protein, 967 aa, Homo sapiens
MQRAVPEGFGRRKLGSDMGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGAQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 60 - protein, 890 aa, Homo sapiens [sequence omitted]

SEQ ID NO: 61 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 62 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 63 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 64 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 65 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 66 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 67 - protein, 188 aa, Homo sapiens [sequence omitted]

SEQ ID NO: 68 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 69 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 70 - protein, 189 aa, Homo sapiens
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 71 - protein, 189 aa, Homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 72 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 73 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 74 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 75 - protein, 153 aa, Homo sapiens [sequence omitted]

SEQ ID NO: 76 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 77 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 78 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 79 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 80 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 81 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 82 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 83 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 84 - protein, 375 aa, Homo sapiens
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 85 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 86 - protein, 244 aa, Mus musculus
MLPSLIQPCSSGTLLMLLMSNLFLWEKVSSAPINASEAVLSDLKDLFDNATVLSGEMSKLGVIMRKEFFMNSFSSETFNKIILDLHKSTENITKAFNSCHTVPINVPETVEDVRKTSFEEFLKMVLHMLLAWKEPLKHLVTELSALPECPYRILSKAEAIEAKNKDLLEYIIRIISKVNPAIKENEDYPTWSDLDSLKSADKETQFFALYMFSFCLRIDLETVDFLVNFLKCLLLYDDVCYSEF

SEQ ID NO: 87 - protein, 1427 aa, Homo sapiens
MSMLKPSGLKAPTKILKPGSTALKTPTAVVAPVEKTISSEKASSTPSSETQEEFVDDFRVGERVWVNGNKPGFIQFLGETQFAPGQWAGIVLDEPIGKNDGSVAGVRYFQCEPLKGIFTRPSKLTRKVQAEDEANGLQTTPASRATSPLCTSTASMVSSSPSTPSNIPQKPSQPAAKEPSATPPISNLTKTASESISNLSEAGSIKKGERELKIGDRVLVGGTKAGVVRFLGETDFAKGEWCGVELDEPLGKNDGAVAGTRYFQCQPKYGLFAPVHKVTKIGFPSTTPAKAKANAVRRVMATTSASLKRSPSASSLSSMSSVASSVSSRPSRTGLLTETSSRYARKISGTTALQEALKEKQQHIEQLLAERDLERAEVAKATSHVGEIEQELALARDGHDQHVLELEAKMDQLRTMVEAADREKVELLNQLEEEKRKVEDLQFRVEEESITKGDLETQTKLEHARIKELEQSLLFEKTKADKLQRELEDTRVATVSEKSRIMELEKDLALRVQEVAELRRRLESNKPAGDVDMSLSLLQEISSLQEKLEVTRTDHQREITSLKEHFGAREETHQKEIKALYTATEKLSKENESLKSKLEHANKENSDVIALWKSKLETAIASHQQAMEELKVSFSKGLGTETAEFAELKTQIEKMRLDYQHEIENLQNQQDSERAAHAKEMEALRAKLMKVIKEKENSLEAIRSKLDKAEDQHLVEMEDTLNKLQEAEIKVKELEVLQAKCNEQTKVIDNFTSQLKATEEKLLDLDALRKASSEGKSEMKKLRQQLEAAEKQIKHLEIEKNAESSKASSITRELQGRELKLTNLQENLSEVSQVKETLEKELQILKEKFAEASEEAVSVQRSMQETVNKLHQKEEQFNMLSSDLEKLRENLADMEAKFREKDEREEQLIKAKEKLENDIAEIMKMSGDNSSQLTKMNDELRLKERDVEELQLKLTKANENASFLQKSIEDMTVKAEQSQQEAAKKHEEEKKELERKLSDLEKKMETSHNQCQELKARYERATSETKTKHEEILQNLQKTLLDTEDKLKGAREENSGLLQELEELRKQADKAKAAQTAEDAMQIMEQMTKEKTETLASLEDTKQTNAKLQNELDTLKENNLKNVEELNKSKELLTVENQKMEEFRKEIETLKQAAAQKSQQLSALQEENVKLAEELGRSRDEVTSHQKLEEERSVLNNQLLEMKKRESKFIKDADEEKASLQKSISITSALLTEKDAELEKLRNEVTVLRGENASAKSLHSVVQTLESDKVKLELKVKNLELQLKENKRQLSSSSGNTDTQADEDERAQESQIDFLNSVIVDLQRKNQDLKMKVEMMSEAALNGNGDDLNNYDSDDQEKQSKKKPRLFCDICDCFDLHDTEDCPTQAQMSEDPPHSTHHGSRGEERPYCEICEMFGHWATNCNDDETF

SEQ ID NO: 88 - protein, 104 aa, Homo sapiens
MSSAARSRLTRATRQEMLFLALLLLPVVVAFARAEAEEDGDLQCLCVKTTSQVRPRHITSLEVIKAGPHCPTAQLIATLKNGRKICLDLQALLYKKIIKEHLES

SEQ ID NO: 89 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 90 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIGPVALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGFCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 91 - protein, 956 aa, Homo sapiens
METQELRGALALLLLCFFTSASQDLQVIDLLTVGESRQMVAVAEKIRTALLTAGDIYLLSTFRLPPKQGGVLFGLYSRQDNTRWLEASVVGKINKVLVRYQREDGKVHAVNLQQAGLADGRTHTVLLRLRGPSRPSPALHLYVDCKLGDQHAGLPALAPIPPAEVDGLEIRTGQKAYLRMQGFVESMKIILGGSMARVGALSECPFQGDESIHSAVTNALHSILGEQTKALVTQLTLFNQILVELRDDIRDQVKEMSLIRNTIMECQVCGFHEQRSHCSPNPCFRGVDCMEVYEYPGYRCGPCPPGLQGNGTHCSDINECAHADPCFPGSSCINTMPGFHCEACPRGYKGTQVSGVGIDYARASKQVCNDIDECNDGNNGGCDPNSICTNTVGSFKCGPCRLGFLGNQSQGCLPARTCHSPAHSPCHIHAHCLFERNGAVSCQCNVGWAGNGNVCGTDTDIDGYPDQALPCMDNNKHCKQDNCLLTPNSGQEDADNDGVGDQCDDDADGDGIKNVEDNCRLFPNKDQQNSDTDSFGDACDNCPNVPNNDQKDTDGNGEGDACDNDVDGDGIPNGLDNCPKVPNPLQTDRDEDGVGDACDSCPEMSNPTQTDADSDLVGDVCDTNEDSDGDGHQDTKDNCPQLPNSSQLDSDNDGLGDECDGDDDNDGIPDYVPPGPDNCRLVPNPNQKDSDGNGVGDVCEDDFDNDAVVDPLDVCPESAEVTLTDFRAYQTVVLDPEGDAQIDPNWVVLNQGMEIVQTMNSDPGLAVGYTAFNGVDFEGTFHVNTVTDDDYAGFLFSYQDSGRFYVVMWKQTEQTYWQATPFRAVAQPGLQLKAVTSVSGPGEHLRNALWHTGHTPDQVRLLWTDPRNVGWRDKTSYRWQLLHRPQVGYIRVKLYEGPQLVADSGVIIDTSMRGGRLGVFCFSQENIIWSNLQYRCNDTVPEDFEPFRRQLLQGRV

SEQ ID NO: 92 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 93 - protein, 227 aa, Homo sapiens
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 94 - protein, 365 aa, Homo sapiens
MPGGKKVAGGGSSGATPTSAAATAPSGVRRLETSEGTSAQRDEEPEEEGEEDLRDGGVPFFVNRGGLPVDEATWERMWKHVAKIHPDGEKVAQRIRGATDLPKIPIPSVPTFQPSTPVPERLEAVQRYIRELQYNHTGTQFFEIKKSRPLTGLMDLAKEMTKEALPIKCLEAVILGIYLTNSMPTLERFPISFKTYFSGNYFRHIVLGVNFAGRYGALGMSRREDLMYKPPAFRTLSELVLDFEAAYGRCWHVLKKVKLGQSVSHDPHSVEQIEWKHSVLDVERLGRDDFRKELERHARDMRLKIGKGTGPPSPTKDRKKDVSSPQRAQSSPHRRNSRSERRPSGDKKTSEPKAMPDLNGYQIRV

SEQ ID NO: 95 - protein, 187 aa, Homo sapiens
MPEVERKPKITASRKLLLKSLMLAKAKECWEQEHEEREAEKVRYLAERIPTLQTRGLSLSALQDLCRELHAKVEVVDEERYDIEAKCLHNTREIKDLKLKVMDLRGKFKRPPLRRVRVSADAMLRALLGSKHKVSMDLRANLKSVKKEDTEKERPVEVGDWRKNVEAMSGMEGRKKMFDAAKSPTSQ

SEQ ID NO: 96 - protein, 182 aa, Homo sapiens
MGDEEKRNRAITARRQHLKSVMLQIAATELEKEESRREAEKQNYLAEHCPPLHIPGSMSEVQELCKQLHAKIDAAEEEKYDMEVRVQKTSKELEDMNQKLFDLRGKFKRPPLRRVRMSADAMLKALLGSKHKVCMDLRANLKQVKKEDTEKERDLRDVGDWRKNIEEKSGMEGRKKMFESES

SEQ ID NO: 97 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 98 - protein, 334 aa, Homo sapiens
MTENSDKVPIALVGPDDVEFCSPPAYATLTVKPSSPARLLKVGAVVLISGAVLLLFGAIGAFYFWKGSDSHIYNVHYTMSINGKLQDGSMEIDAGNNLETFKMGSGAEEAIAVNDFQNGITGIRFAGGEKCYIKAQVKARIPEVGAVTKQSISSKLEGKIMPVKYEENSLIWVAVDQPVKDNSFLSSKVLELCGDLPIFWLKPTYPKEIQRERREVVRKIVPTTTKRPHSGPRSNPGAGRLNNETRPSVQEDSQAFNPDNPYHQQEGESMTFDPRLDHEGICCIECRRSYTHCQKICEPLGGYYPWPYNYQGCRSACRVIMPCSWWVARILGMV

SEQ ID NO: 99 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 100 - protein, 1434 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRFLKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 101 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 102 - protein, 79 aa, Homo sapiens
ASGFRLPEDPSHPRVLIGPGTGIAPFLSFWVQGGRMTPVFECRSPNEDHIYQEEMLEMARKGVLPAVPTAYSCLPGKPK

SEQ ID NO: 103 - protein, 69 aa, Homo sapiens
DPSHPRILVRPGTGIAPFHSFWQQRLHDSQRKVVLEGRRTLVFWCRRPDEDRHYREEMLEMTRKRVLHA

SEQ ID NO: 104 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

(19) United States
(12) Patent Application Publication
(10) Pub. No.: US 2013/0216608 A1
(43) Pub. Date: Aug. 22, 2013

(54) Airway Administration of Angiogenesis Inhibitors

(71) Applicants: Lars Heslet, Gentofte (DK); Kaare Fiala, Frederiksberg (DK)

(72) Inventors: Lars Heslet, Gentofte (DK); Kaare Fiala, Frederiksberg (DK)

(73) Assignee: Trifoilium ApS, Horsholm (DK)

(21) Appl. No.: 13/821,098

(22) PCT Filed: Sep. 9, 2011
(86) PCT No.: PCT/DK2011/050338

§ 371 Date: May 1, 2013

(30) Foreign Application Priority Data
Sep. 9, 2010 (DK) PA 2010 00807

(51) Int. Cl. A61K 31/454; A61K 31/185; A61K 31/404; A61K 39/395

(52) U.S. Cl. CPC A61K 31/454; A61K 39/3955; A61K 31/185; A61K 31/404
USPC 424/450; 514/323; 424/133.1; 514/577; 514/414

(57) ABSTRACT

The present invention provides methods for the local pulmonary treatment of lung metastases and/or primary lung cancer, such as squamous lung cancer and/or small cell lung cancer, using local deposition of one or more agents capable of inhibiting angiogenesis and/or an agent that acts as an active anti-cancer agent and/or an active anti-metastatic agent. The agent capable of inhibiting angiogenesis and/or which acts as an active anti-cancer agent and/or an active anti-metastatic agent is administered to a patient with cancer as a treatment of said metastasis and/or cancer or to extend the lifespan for said cancer patient with reduced systemic side-effects compared to other treatment forms. The agent capable of inhibiting angiogenesis and/or which acts as an active anti-cancer agent and/or an active anti-metastatic agent is administered intratracheal, intrabronchial, intraalveolar or broncioalveolar. Administration for example via inhalation of an aerosol or as a dry powder and/or administered as a bronchoalveolar lavage (BAL) an effective amount of the agent.

All patent and non-patent references cited in the application, or in the present application, are also hereby incorporated by reference in their entirety.

### FIELD OF INVENTION

The present invention relates to compositions comprising an effective amount of one or more agents capable of inhibiting angiogenesis for use as a medicament for the local inhibition of metastasis in pulmonary parenchyma, i.e. in the terminal lung unit (TLU) in particular the alveolar and/or bronchoalveolar space and/or the small airways and/or the bronchioli and/or the alveolar cells including the alveolar macrophages and the pulmonary interstium and pulmonary parenchyma. The composition is particularly useful in the pulmonary parenchyma after inhalation into alveolar and/or bronchoalveolar space and/or the small airways and/or the bronchioli.

### BACKGROUND OF INVENTION

### Cancer Metastasis

Metastasis is the spread of a disease from one organ or part to another non-adjacent organ or part. Only malignant tumor cells and infections have the capacity to metastasize. The majority of cancer deaths are associated with metastatic disease.

Cancer metastases are strongly correlated with a poor prognosis. The multi-step process of metastasis includes release of malignant cells from the primary neoplasm, migration of cancer cells into circulation (hematogenouis spread), interaction with platelets and leukocytes in circulation, adhesion to endothelium at distant sites and growth of the disseminated cancer cells within the vessels or within the tissue following extravasation. Each step in this process requires different types of interaction between cancer cells and the host microenvironment.

When tumor cells metastasize, the new tumor is called a secondary of metastatic tumor and its cells are like those in the original tumor. This means for example that if a breast cancer spreads (metastasizes) to the lung, the secondary tumor is made up of abnormal breast cells, not of abnormal lung cells. The tumor in the lung is thus denoted metastatic breast cancer not lung cancer.

The cells in the metastatic tumor resemble those in the primary tumor. Once the cancerous tissue is examined under a microscope to determine the cell type, a doctor can usually tell whether that type of cell is normally found in the part of body from which the tissue sample was taken.

The lungs are major sites for many cancer metastases. Pulmonary metastases are common and most frequently occur with tumors that have rich systemic venous drainage. Examples of such metastases include but not limited to renal cancers, bone sarcomas, choriocarcinomas, melanomas, testicular teratomas, and thyroid carcinomas. Most pulmonary metastases arise from common tumors, such as breast, colorectal, prostate, bronchial, head-and-neck, and renal cancers.

TABLE 1
Incidence of Pulmonary Metastases According to Site

| | Frequency at | Frequency at |
|---|---|---|
| Primary Tumor | Presentation, % | Autopsy, % |
| Choriocarcinoma | 60 | 70-100 |
| Melanoma | 5 | 66-80 |
| Testis, germ cell | 12 | 70-80 |
| Osteosarcoma | 15 | 75 |
| Thyroid | 7 | 65 |
| Kidney | 20 | 50-75 |
| Head and neck | 5 | 15-40 |
| Breast | 4 | 60 |
| Bronchus | 30 | 40 |
| Colorectal | <5 | 25-40 |
| Prostate | 5 | 15-50 |
| Bladder | 7 | 25-30 |
| Uterus | <1 | 30-40 |
| Cervix | <5 | 20-30 |
| Pancreas | <1 | 25-40 |
| Esophagus | <1 | 20-35 |
| Stomach | <1 | 20-35 |
| Ovary | 5 | 10-25 |
| Hepatoma | <1 | 20-60 |

(Hassan I. Lung Metastasis, e-medicine, 25/10/2006 http://www.emedicine.com/RADIO/topic404.htm)

Angiogenesis is a biological process of generation new blood vessels into a tissue or organ. Under normal physiological conditions, humans and animals undergo angiogenesis only in very specific restricted situations. For example, angiogenesis is normally observed in wound healing, fetal and embryonal development and formation of the corpus luteum, endometrium and placenta. It has been reported that new vessel growth is tightly controlled by angiogenic regulators and the switch of the angiogenesis phenotype depends on the net balance between up-regulation of angiogenic stimulators and down-regulation of angiogenic suppressors.

Angiogenesis is a normal process in growth and development, as well as in wound healing as described above. However, this is also a fundamental step in the transition of tumors from a small size with supply of substrate to larger size tumors and/or a growing metastasis, which needs its own vascular supply to continued growth.

Angiogenesis occurs stepwise as follows: vasodilation and increased permeability of preexisting vessels, decomposition of a basement membrane by protease produced by activated vascular endothelial cells, migration and proliferation of the vascular endothelial cells, tube formation of the vascular endothelial cells, formation of the basement membrane and encirclement of peripheral cells and finally the differentiation and maturation of blood vessels.

Angiogenesis may be caused by various proliferation factors, cytokines, arachidonic acid metabolites, monobutyrin and the like with the proliferation factors considered most important. For angiogenesis to occur, pro-angiogenic factors must outweigh anti-angiogenic factors.

Angiogenesis is closely related to various diseases particularly diabetic retinopathy, retinopathy of prematurity, macular degeneration, neovascular glaucoma, retinal vein occlusion, retinal artery occlusion, pterygium, rubeosis, corneal neovasulature, solid tumors, hemangioma, proleration and transfer of tumors and the like.

Vasculogenesis is the term used for spontaneous blood-vessel formation and intussesception is the term for new blood vessel formation by splitting off existing ones. Neovascularization allows tumor progression to ensue. With angiogenesis, the tumor becomes invasive locally and systemically.

The modern clinical application of the principle “angiogenesis” can be divided into two main areas; anti-angiogenic therapies and pro-angiogenic therapies. Whereas anti-angiogenic therapies are trying to fight cancer and malignancies, the pro-angiogenic therapies are becoming more and more important in the search for new treatments for cardiovascular diseases.

An angiogenesis inhibitor is a substance that inhibits angiogenesis. It can be endogenous or administered from outside as drug or a dietary component.

### VEGF

The major mediator of tumor angiogenesis is vascular endothelial growth factor A (VEGF-A, also called VEGF). VEGF signals through the VEGF receptor 2 (VEGFR-2), which mediates sprouting angiogenesis. VEGFR-2 is also called kinase-insert domain-containing receptor (KDR) in humans and fetal liver kinase 1 (flk-1) in mice. VEGF is expressed in most types of human cancer, and increased expression in tumors is often associated with a less favorable prognosis. Induction of VEGF expression in tumors may be caused by factors such as hypoxia, low pH, inflammatory cytokines (e.g. interleukin-6), growth factors (e.g. basic fibroblast growth factor), sex hormones (both androgens and estrogens), and chemokines (e.g. stromal-cell-derived factor 1).

The binding of VEGF to VEGFR-2 activates a cascade of signaling events resulting in the up-regulation of genes mediating proliferation and migration of endothelial cells, promoting their survival as well as vascular permeability. The VEGFR-2 receptor dimerizes upon binding of VEGF, which is followed by intracellular activation of the PLCγ-PKC-Raf kinase-MEK-mitogen-activated protein kinase (MAPK) pathway and subsequent initiation of DNA synthesis and cell growth, whereas activation of the phosphatidylinositol 3′-kinase (PI3K)-Akt pathway leads to increased endothelia)-cell survival. Activation of src can lead to actin cytoskeleton changes and induction of cell migration. VEGF receptors are located on the endothelial-cell surface; however, intracellular (“intracrine”)-signaling VEGF receptors (VEGFR-2) may be present as well, and they are involved in promoting the survival of endothelial cells. The detailed structure of the intracellular VEGFR-2 in endothelial cells is not yet known, but it is shown as the full-length receptor that is normally bound to the cell surface. Binding of VEGF-C to VEGFR-3 mediates lymphangiogenesis. VEGF165 can bind to neuropilin (NRP) receptors, which can act as coreceptors with VEGFR-2 (horizontal arrow) to regulate angiogenesis. EGFR denotes epidermal growth factor receptor, flt-1 fms-like tyrosine kinase 1, PlGF placental growth factor, PTEN phosphatase and tensin homologue, S—S disulfide bond, and VHL von Hippel-Lindau.

### SUMMARY OF INVENTION

A first aspect of the invention is to provide a composition for airway administration to a subject comprising an effective amount of one or more agents capable of inhibiting angiogenesis for use as a medicament for the local inhibition of metastasis in pulmonary parenchyma.

Another aspect of the present invention relates to the use of a composition for airway administration comprising an effective amount of one or more agents capable of inhibiting angiogenesis for use as a medicament for the local inhibition of metastasis in the pulmonary parenchyma.

Yet another aspect, the present invention is to provide a composition for airway administration comprising an effective amount of one or more agents capable of inhibiting angiogenesis for use as a medicament for the local inhibition of metastasis in the pulmonary parenchyma.

Another aspect of the present invention relates to a method for local inhibition of metastasis of the pulmonary parenchyma in a human subject comprising airway administration of an effective amount of one or more agents capable of inhibiting angiogenesis.

The airway administration of one or more angiogenesis inhibitors can be given alone or as a supplement to systemic administration of the same or other drugs including but not limited to active anti-cancer agents chemotherapeutics.

### DESCRIPTION OF DRAWINGS

FIG. 1. The difference between A) systemic drug administration, B) systemic pulmonary drug administration and C) local pulmonary drug deposition.

### DETAILED DESCRIPTION OF THE INVENTION

The present invention relates to the airway administration, by any appropriate method including, but not limited to, intratracheal, intrabronchial, bronchio-alveolar or intraalveolar administration, to a human subject inclusive of both adults and children, of one or more agents capable of inhibiting angiogenesis and/or active anti-cancer agents and/or anti-metastatic agents, however prepared, to prevent or reduce the formation of new blood vessels, particularly in connection with metastasis in the lungs and/or to extend the lifespan for patients with pulmonary metastasis or any type of cancer of the lungs with reduced systemic side-effects compared to other treatment form. This may be achieved by the inhalation of one or more agents capable of inhibiting angiogenesis and/or active anti-cancer agents and/or anti-metastatic agents as an aerosolized or nebulized solution or suspension or inhaled powder or gel, with or without added stabilizers or other excipients.

Using pulmonary deposition of one or more agents capable of inhibiting angiogenesis and/or active anti-cancer agents and/or anti-metastatic agent a significant spill over of the drug to the systemic circulation is circumvented or at least significantly reduced. In this way, for example by using inhalation administration, systemic adverse effects may be eliminated or reduced. The many anti-angiogenetic drugs have multiple adverse effects (AE) using systemic administration. These AEs include but are not limited to sedation/somnolence, fatigue, depression tremor, headache, cardiovascular, thrombosis, bradycardia, hypotension, orthostatic hypotension, edema, rash, pruritus, dermatitis, gastrointestinal adverse effects with constipation and xerostomia and endocrine AE with subclinical hypothyroidism. In many studies anti-angiogenetic drugs have consequently been withdrawn or the dose reduced due to significant adverse effects.

The lungs have a major potential for pro-angiogenesis, which is a prerequisite for further growth enhancement, from the early pulmonary nidation of cancer cells with further growth of hematogeneous spread of tumors to form larger metastases as a pulmonary metastatic disease. Using the pulmonary deposition of one or more agents capable of inhibiting angiogenesis and/or acting as an active anti-cancer agent and/or an active anti-metastatic agent, the well-described multiple and serious adverse effects of antiangiogenetic drugs may be totally avoided, due to lack of or reduced spill over from the lung compartment to the systemic circulation. Here the neurologic adverse effects of angiogentic inhibitors are far the most important toxic.

### DEFINITIONS

An agent capable of inhibiting angiogenesis may be any agent capable of inhibiting angiogenesis and/or any active anti-cancer agents and/or any anti-metastatic agents.

Amino Acid Residue: That part of the amino acid which is present in the polypeptide chain in which the amino acid is linked to other amino acids by peptide (amide) bonds. The amino acid residues described herein are preferably in the “L” isomeric form. However, the amino acid encompasses every amino acid such as L-amino acid, D-amino acid, alpha-amino acid, beta-amino acid, gamma-amino acid, natural amino acid and synthetic amino acid or the like, as long as the desired functional property is retained by the polypeptide. Further included are natural or synthetic amino acids which have been modified. NH₂ refers to the free amino group present at the amino terminus of a polypeptide. COOH refers to the free carboxy group present at the carboxy terminus of a polypeptide. Standard polypeptide abbreviations for amino acid residues are used herein.

It should be noted that all amino acid residue sequences represented herein by formulae have a left-to-right orientation in the conventional direction of amino terminus to carboxy terminus. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Modified amino acid: an amino acid wherein an arbitrary group thereof is chemically modified. In particular, a modified amino acid chemically modified at the alpha-carbon atom in an alpha-amino acid is preferable.

Angiogenesis: The growth of new blood vessels.

Angiogenesis stimulator: A substance that stimulates angiogenesis.

Angiogenesis inhibitor: When used herein the term “Angiogenesis inhibitor” is meant to describe a substance that inhibits angiogenesis or an anti-angiogenic substance. These terms may be used interchangeably.

Anti-angiogenic: an inhibitor of angiogenesis

ECM: Extracellular matrix

Metastasis: The process of spread of a disease from one organ or part to another non-adjacent organ or part.

Neovasculature The new network of blood vessels (of a tumor).

Polypeptide: The phrase polypeptide refers to a molecule comprising amino acid residues which do not contain linkages other than amide linkages between adjacent amino acid residues. The phrase peptide is used accordingly.

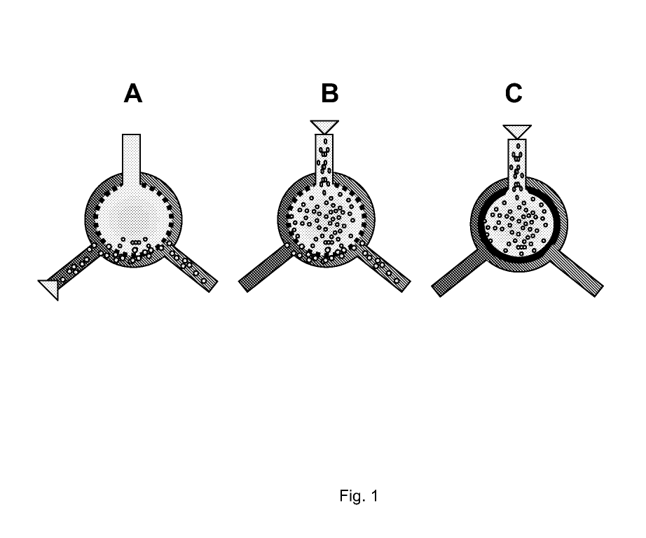

Pulmonary deposition of drug: When used herein the term “deposition of drugs in the lungs” and other variations on this theme is meant to describe local pulmonary drug administration and not systemic drug administration via the airways as visualized in FIG. 1. Pulmonary deposition or delivery of a local dose thus refers to topical administration to the lung for diseases of the lungs. Alternatively, a systemic dose typically describes administration via the lung for absorption from the alveolar region to the circulation to treat systemic disorders, such as diabetes, migraine, osteoporosis, and hormone regulation

Primary cancer: Cancer is generally classified according to the tissue from which the cancerous cells originate the primary cancer/tumor.

Pro-angiogenic: a stimulator of angiogenesis

RGD motif/sequence: This tripeptide motif (Arg-Gly-Asp) can be found in proteins of the extracellular matrix. Integrins link the intracellular cytoskeleton of cells with the extracellular matrix by recognizing this RGD motif. Without attachment to the extracellular matrix, cells normally undergo apoptosis ('anoikis').

Splitting angiogenesis or intussusception: a type of angiogenesis where the capillary wall extends into the lumen to split a single vessel in two.

Sprouting angiogenesis: the formation of new vessels by four steps: First angiogenic growth factors activate receptors present on endothelial cells present in pre-existing veins. Second, the activated endothelial cells release proteases that degrade the basement membrane in order to allow endothelial cells to escape from the original (parent) vessel walls. The endothelial cells then proliferate into the surrounding matrix and form solid sprouts connecting neighboring vessels. As sprouts extend toward the source of the angiogenic stimulus, endothelial cells migrate in tandem, using integrins. These sprouts form loops to become a full-fledged vessel lumen as cells migrate to the site of angiogenesis.

### Metastasis

One aspect of the present invention relates to a method of inhibiting the formation of new blood vessels. Thus, the present invention relates to the treatment of individuals suffering from, or at risk of suffering from, cancer and/or metastasis.

The metastasis to be treated with the angiogenesis inhibitors of the present invention may have spread from any primary site cancer including but not limited to Breast Cancer, Colorectal Cancer, Prostate Cancer, Stomach Cancer, Ovarian Cancer, Kidney Cancer, spleen cancer, Malignant Melanoma, Esophageal Cancer, Head and Neck Cancers, Testis/germ cell Cancer, bladder cancer, cancer of the uterus, hepatoma, pancreatic cancer, cervix cancer, osteosarcoma, thyroid cancer, bronchus cancer, Choriocarcinoma and/or metastases without a primary tumor found.

Administration of an effective amount of one or more agents capable of inhibiting angiogenesis and/or active anti-cancer agents and/or anti-metastatic agents thereof via intratracheal, intrabronchial, intraalveolar or bronchio-alveolar administration, either as the sole anticancer intervention but also combined with systemic anticancer therapy is particularly useful in the prevention of formation of new blood vessels (angiogenesis) for example in connection with metastasis and/or cancer.

### Cancer

Cancerous diseases are scientifically designated neoplasia or neoplasms and may be benign or malignant. Cancers are classified by the type of cell that resembles the tumor and, therefore, the tissue presumed to be the origin of the tumor. The following general categories are applied:

Carcinoma: malignant tumors derived from epithelial cells. This group includes the most common cancers, comprising the common forms of breast, prostate, lung and colon cancer.

Lymphoma and Leukemia: malignant tumors derived from blood and bone marrow cells.

Sarcoma: malignant tumors derived from connective tissue, or mesenchymal cells

Mesothelioma: tumors derived from the mesothelial cells lining the peritoneum and the pleura.

Glioma: tumors derived from glia, the most common type of brain cell

Germinoma: tumors derived from germ cells, normally found in the testicle and ovary.

Choriocarcinoma: malignant tumors derived from the placenta.

In a preferred embodiment, the agent capable of inhibiting angiogenesis and/or acts as an active anti-cancer agent and/or an active anti-metastatic agent is administered to a patient with cancer as a treatment or to cause regression of said cancer or to extend the lifespan for said cancer patient with reduced systemic side-effects compared to other treatment forms.

In preferred embodiments of the invention the cancer is a cancer of the lungs including but not limited to primary lung cancers of epithelial origin i.e. lung carcinomas, lung sarcoma, lung carcinoid, Small cell lung carcinoma (SCLC) or Non-small cell lung carcinoma (NSCLC) including but not limited to Squamous cell lung carcinoma, large cell carcinoma, giant cell and spindle cell carcinoma, Adenocarcinoma including but not limited to Adenocarcinoma (not otherwise specified), Bronchioloalveolar carcinoma, Adenosquamous carcinoma, Papillary adenocarcinoma, Mucoepidermoid carcinoma, Adenoid cystic carcinoma or other specified adenocarcinoma; or other/unspecified non-small cell lung carcinoma or any lung cancer of unknown etiology.

### Angiogenesis Inhibitors

In one preferred embodiment of the present invention, an agent is administered to the subject, said agent being capable of inhibiting angiogenesis and/or acts as an active anti-cancer agent and/or an active anti-metastatic agent. The administration of said agent may extend the lifespan for said cancer patient with reduced systemic side-effects compared to other treatment forms

The agent capable of exhibiting the mentioned effect may be any type of agent, for example, the agent may be selected from the group comprising proteins, peptides, polypeptides, antibodies or an antigen-binding fragment thereof, peptide-like antibodies, antisense-RNA, antisense-DNA, siRNA, other polynucleotides, or organic molecules. The antibody or functional equivalent thereof may be any type of antibody known in the art, for example a polyclonal or a monoclonal antibody derived from a mammal or a synthetic antibody, such as a single chain antibody or hybrids comprising antibody fragments for example directed against a promoter or inducer of angiogenesis. In addition functional equivalents of antibodies may be antibody fragments, in particular epitope binding fragments. Furthermore, antibodies or functional equivalent thereof may be small molecule mimicking an antibody. Naturally occurring antibodies are immunoglobulin molecules consisting of heavy and light chains. Functional equivalents of antibodies may be a fragment of an antibody, preferably an antigen binding fragment or a variable region. Examples of antibody fragments useful with the present invention include Fab, Fab′, F(ab′)₂ and Fv fragments. The antibody can also be a single chain antibody (“SCA”), defined as a genetically engineered molecule containing the variable region of the light chain, the variable region of the heavy chain, and linked by a suitable polypeptide linker as a genetically fused single chain molecule. Such single chain antibodies are also referred to as “single-chain Fv” or “sFv” antibody fragments.

Anti-angiogenic strategies according to the present invention may include but are not limited to inhibition of angiogenic stimulus, amplification of endogenous suppressors of angiogenesis and endothelial cell proliferation.

An anti-angiogenic agent according to the present invention may for example be an agent capable of Inhibition of pro-angiogenic factors.

The VEGF family is best characterized of the pro-angiogenic growth factors. VEGF family consists of VEGF-A, VEGF-B, VEGF-C, VEGF-D, VEGF-E and placenta growth factor (PlGF). VEGF-A is considered the most potent and specific of the many pro-angiogenic factors. Blood vessel development is dependent upon VEG family of proteins and their receptors.

VEGF is expressed in most types of human cancer, and increased expression in tumors often correlates with the extent of angiogenesis and prognosis. Induction of VEGF expression in tumors may be caused by factors such as hypoxia, low pH, inflammatory cytokines (e.g. interleukin-6), growth factors (e.g. basic fibroblast growth factor), sex hormones (both androgens and estrogens), and chemokines (e.g. stromal-cell-derived factor 1). VEGF promotes survival and proliferation of endothelial cells, increase vascular permeability.

Vascular Endothelial Growth Factor (VEGF) may thus be an appealing target for an anti-angiogenic agent according to the present invention. In particular embodiments the anti-angiogenic agent according to the invention may be an inhibitor of a VEGF family member. One such anti-angiogenic agent is the recombinant humanized monoclonal IgG1 antibody Bevacizumab/Avastin (see table 1 below). Bevacizumab/Avastin recognizes all isoforms of VEGF1. In one embodiment the anti-angiogenic agent according to the invention may be Bevacizumab.

Anti-angiogenesis targets may include agents targeting the neovasculature which may include but are not limited to proteases that breakdown the ECM, growth factors that stimulate endothelial cell proliferation, integrins that allow adhesion of endothelial cells, endothelial cell apoptosis.

Agents capable of inhibiting ECM Breakdown may include inhibitors of metalloproteinases (MMPs) which are proteolytic enzymes that cleave the basement membrane. Thus, in one embodiment an agent capable of inhibiting angiogenesis according to the present invention may be an MMP inhibitor including but not limited to Marimastat and Batimastat (see Table 2 below).

Tumor cells are hypoxic, which induces hypoxia-inducible factor 1 (HIF1) to signal over production of growth factors. Cell growth may be inhibited by targeting growth factors such as VEGF, PDGF, bFGF, IL-8 or by targeting the growth factor receptors. Thus, in one embodiment an agent capable of inhibiting angiogenesis according to the present invention may be an inhibitor of any of the above-mentioned stimulators of angiogenesis.

Drugs that prevent cell proliferation are also useful in some embodiments of the present invention as an anti-angiogenic agent. Such drugs may include Suramin (see table 1 below) which prevents bFGF and VEGF from binding to the active site of their receptors through competitive inhibition, the Bevacizumab/Avastin (see table 1 below) antibody that targets VEGF (binds to VEGFa to inhibit VEGFR1 and VEGFR2) and thus enables normalization comprising reduced blood vessel permeability and interstitial pressure and angiostatin (see table 1 below) which binds to HGF (hepatocyte growth factor) and blocks endothelial cell surface ATP-synthase.

In one embodiment the anti-angiogenic agent according to the invention may be an agent that inhibits cell adhesion. This may for example be achieved by targeting integrin α_{V}β₃ by for example antibodies against α_{V}β₃ ligands, α_{V}β₃ antagonists and siRNA or antisense RNA directed against α_{V}β₃. An α_{V}β₃ antagonist according to the present invention may be Cilengitide (see table 1 below) which contains the RGD (Arg-Gly-Asp) sequence and blocks the ligand from binding to the integrin. Any soluble RGD containing peptides or antibodies may be used as an anti-angiogenic agent according to the present invention. RGD containing peptides or antibodies induce apoptosis, inhibit cell attachment, and consequently induce apoptosis.

In another embodiment the anti-angiogenic agent may be an agent capable of inducing apoptosis, including but not limited to tumor necrosis factor (TNF) for example causes endothelial cell apoptosis in tumor cells and induces inflammation and endothelial cell growth in normal cells. Another target may be to down-regulate or block the interactions of Bcl-2 with pro-apoptotic proteins such as endostatin and angiostatin.

In another embodiment the anti-angigenic agent may be Celecoxib (see table 2 below) which is a COX-2 (cyclooxygenase-2) Inhibitor. Celecoxib decrease vascular permeability, endothelial cell proliferation, endothelial cell migration, MMP production and affect the integrin signaling pathway.

Anti-angiogenesis targets may include agents targeting preexisting vasculature such as various vasculature targeting agents (VTAs). VTAs disrupt already-present blood vessels such as Combretastatin A-4 (prodrugs: CA4P and Oxigene), which destabilizes microtubules of vascular cells and DMXAA (a flavonoid analog) that increases NF-kb transcription by phosphorylation leading to the production of proteins that change vascular cell shape and organization eventually leading to apoptosis of these cells (see table 1 below).

In a preferred embodiment of the invention the anti-angigenic agent is thalidomide or an analogue or derivative thereof. Thalidomide has been approved in 2006 for combination therapy with dexamethasone for treatment of multiple myeloma (cancer of plasma cells). Thalidomide block bFGF and VEGF, inhibits COX-2 and interferes with Tumor Necrosis Factor-α.

Two major classes of thalidomide analogues/derivatives have been identified: selective cytokine inhibitory drugs (SelCIDs) and Immunomodulatory imide Drugs (IMiDs). IMiDs include but are not limited to C-5013 or lenalidomide, CC-4047 or Actimid, ENMD-0995 and IMiD3. IMiDs are amino-phtaloyl-substituted thalidomide analogues i.e. 4-amino analogues, in which an amino group is added to the fourth carbon of the phtaloyl ring of thalidomide. IMiDs inhibit endothelial cell migration and adhesion, maybe due to down-regulation of integrins.

SelCIDs includes but are not limited to SelCID-3. Other analogues that have been synthesized based on thalidomes metabolites include but are not limited to CPS 11 (N-substituted analogue), CPS 45 and CPS 49 (tetraflourinated analogues).

Other analogues include but are not limited to phthalimidophthalimide and EM-12.

Analogues and derivatives of thalidomide may be assayed for the anti-angiogenic activity using several different assays including but not limited to an assay to determine the potency to inhibit TNF-α in lipopolysaccharide (LPS) stimulated human peripheral blood mononuclear cell (PBMC) bioassays (Muller G W, Chen R, Huang S Y et al., Bioorg Med Chem Lett, 1999, 9: 1625-1630) or the human umbilical vein endothelial cell (HUVEC) proliferation and tube formation assay (as described http://www.rndsystems.com/bioassay_detail_objectname_HUVECS.aspx) or in Wakasugi K et al., Proc Natl Acad Sci USA. 2002 Jan. 8; 99(1): 173-177) or the rat aortic ring assay (Ng S S et al., Cancer Res 2003, 63:3189-94).

Other analogues of thalidomide according to the present invention include those described in US2008167272 or WO02068414 or WO03014315 or WO2005016326 or EP0856513 or US 2004/007685

Thus, an agent capable of inhibiting angiogenesis and/or an active anti-cancer and/or an anti-metastatic agent according to the present invention may be any angiogenesis inhibitor such as an endogenous angiogenesis inhibitor including but not limited to VEGFR-1, NRP-1, angiopoitin 2, TSP-1, TSP-2, angiostatin, endostatin, vasostatin, calreticulin, platelet factor-4, TIMP, CDAI, Meth-1, Meth-2, IFN-α, -β and -γ, CXCL10, IL-4, -12 and -18, prothrombin (kringle domain-2), antithrombin III fragment, prolactin, VEGI, SPARC, osteopontin, maspin, canstatin, proliferin-related protein and restin as listed in Table 1.

An agent capable of inhibiting angiogenesis and/or an active anti-cancer and/or an anti-metastatic agent according to the present invention may also be any angiogenesis inhibitor such as a known angiogenic drug including but not limited to bevacizumab/Avastin, carboxyamidotriazole, TNP-470, CM101, IFN-α, IL-12, platelet factor-4, suramin, SU5416, thrombospondin, VEGFR antagonists, angiostatic steroids, heparin, Cartilage-Derived Angiogenesis Inhibitory Factor, thalidomide and derivates and/or analogues thereof. SelCIDs, IMiDs including but not limited to C-5013/lenalidomide, CC-4047/Actimid, ENMD-0995 and IMiD3, thalidomes metabolites including but not limited to CPS 11 (N-substituted analogue), CPS 45 and CPS 49 (tetraflourinated analogues) or phthalimidophthalimide and EM-12, matrix metalloproteinase inhibitors, angiostatin, endostatin, 2-methoxyestradiol, tecogalan, thrombospondin, prolactin, intergrin α_{V}β₃ inhibitors and linomide as listed in Table 1.

TABLE 2
Overview of agents with an antiangiogentic effect

| Inhibitors | Mechanism | ADDITIONAL INFO |
|---|---|---|
| Soluble vascular endothelial | Decoy receptors for VEGF-B and PIGF | SEQ ID NO 14 (VEGFR 1) |
| growth factor receptor | | SEQ ID NO 15 (VEGFR 2) |
| (VEGFR) such as VEGF- | | SEQ ID NO 16 (VEGFR 3) |
| TRAP | | |
| Neuropilin 1 or 2 | Decoy receptors for VEGF-B and PIGF | SEQ ID NO 17 (Neuropilin-1) |
| (NRP-1 or 2) | | SEQ ID NO 18 (Neuropilin-2) |
| Angiopoietin 2 | Antagonist of angiopoietin 1 | SEQ ID NO 49 |
| Angiostatin (plasminogen) | Inhibit cell proliferation and induce apoptosis | SEQ ID NO 50 |
| | of endothelial cells | |
| Endostatin | Inhibit cell migration, cell proliferation and | SEQ ID NO 51 and 52 |
| | survival of endothelial cells | |
| Vasostatin (Chromogranin-A) | Inhibit cell proliferation of endothelial cells | SEQ ID NO 53 |
| Calreticulin | Inhibit cell proliferation of endothelial cells | SEQ ID NO 54 |
| TIMP (Tissue inhibitor of | Inhibit cell proliferation of endothelial cells | SEQ ID NO 55 (TIMP1) |
| metalloproteinases) | | SEQ ID NO 56 (TIMP2) |
| | | SEQ ID NO 57 (TIMP3) |
| | | SEQ ID NO 58 (TIMP4) |
| Meth-1 (ADAMTS-1) | Mediates the release of antiangiogenic | SEQ ID NO 59 |
| | polypeptides from TSP1 and 2, inhibits | |
| | endothelial cell proliferation by direct | |
| | binding and sequestration of VEGF | |
| Meth-2 (ADAMTS-8) | | SEQ ID NO 60 |
| IFN-α | Inhibit cell migration of endothelial cells, | SEQ ID NO 61 (IFN alpha 21) |
| | downregulate bFGF, downregulate | SEQ ID NO 62 (IFN alpha 10) |
| | angiogenesis stimulators | SEQ ID NO 63 (IFN alpha 14) |
| | | SEQ ID NO 64 (IFN alpha 16) |
| | | SEQ ID NO 65 (IFN alpha 17) |
| | | SEQ ID NO 66 (IFN alpha 1/13) |
| | | SEQ ID NO 67 (IFN alpha 2) |
| | | SEQ ID NO 68 (IFN alpha 4) |
| | | SEQ ID NO 69 (IFN alpha 6) |
| | | SEQ ID NO 70 (IFN alpha 7) |
| | | SEQ ID NO 71 (IFN alpha 8) |
| | | SEQ ID NO 61 (IFN alpha 21) |
| IFN-β | Inhibit cell migration of endothelial cells, | SEQ ID NO 72 |
| | downregulate bFGF | |
| IFN-γ | Inhibit cell migration of endothelial cells, | SEQ ID NO 73 |
| | downregulate bFGF | |
| CXCL10 (C-X-C motif | Inhibit cell migration of endothelial cells, | SEQ ID NO 74 |
| chemokine 10) | downregulate bFGF | |
| Interleukin-4 (IL-4) | Inhibit cell migration of endothelial cells, | SEQ ID NO 75 |
| | downregulate bFGF | |
| Interleukin-12 (IL-12) | inhibit cell migration of endothelial cells, | SEQ ID NO 76 (alpha subunit) |
| | downregulate bFGF, stimulate angiogenesis | SEQ ID NO 77 (beta subunit) |
| | inhibitor formation | |
| Interleukin-18 (IL -18) | inhibit cell migration of endothelial cells, | SEQ ID NO 78 |
| | downregulate bFGF | |
| Prothrombin (kringle | Inhibit cell proliferation of endothelial cells | SEQ ID NO 79 |
| domain-2) | | |
| Antithrombin III fragment | Inhibit cell proliferation of endothelial cells | SEQ ID NO 80 |
| VEGI (Tumor necrosis factor | Affects cell proliferation of endothelial cells | SEQ ID NO 81 |
| ligand superfamily member | | |
| 15, Vascular endothelial cell | | |
| growth inhibitor) | | |
| SPARC (Secreted protein | Inhibit binding and activity of VEGF | SEQ ID NO 82 |
| acidic and rich in cysteine) | | |
| Osteopontin | Inhibit integrin signalling | SEQ ID NO 83 |
| Maspin (serpin B5) | Inhibits proteases | SEQ ID NO 84 |
| Canstatin (Collagen alpha- | Inhibits endothelial cell proliferation and | SEQ ID NO 85 |
| 2(IV) chain) | migration | |
| Proliferin-related protein | A family of peptides with different molecular | SEQ ID NO 86 |
| Prolactin-7D1 precursor, | masses, all containing the N-terminal region | |
| PRP | of PRL. Inhibits endothelial cell migration | |
| Restin, CAP-Gly domain- | Inhibits the migration of endothelial cells | SEQ ID NO 87 |
| containing linker protein 1, | | |
| Cytoplasmic linker protein | | |
| 170 alpha-2, CLIP-170, | | |
| Reed-Sternberg | | |
| intermediate filament- | | |
| associated protein) | | |
| Bevacizumab/Avastin | Inhibits vascular endothelial growth factor | Monoclonal antibody against vascular endothelial |
| | | growth factor |
| Carboxyamidotriazole | Inhibit cell proliferation and cell migration of | |
| | endothelial cells | |
| TNP-470 | Inhibit cell proliferation and cell migration of | A synthetic analog of fumagillin, an antibiotic isolated |
| | endothelial cells | from the fungus Aspergillus fumigatus fresenius |
| CM101 | Activates the immune system | Antiangiogenic polysaccharide derived from group B |
| | | streptococcus |
| Platelet factor-4 | Inhibits binding of angiogenesis stimulators | SEQ ID NO 88 |
| Suramin | Inhibits binding of angiogenesis stimulators | 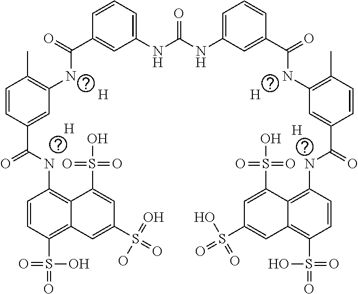 |
| Thrombospondin | Inhibits binding of angiogenesis stimulators | SEQ ID NO 89 (Thrombospondin-1) |
| | | SEQ ID NO 90 (Thrombospondin-2) |
| | | SEQ ID NO 91 (Thrombospondin-3) |
| | | SEQ ID NO 92 (Thrombospondin-4) |
| VEGFR antagonists | Inhibits binding of angiogenesis stimulators | A substance that blocks an enzyme needed to form blood |
| | | vessels. For example the compounds SU5416 (a tyrosine |
| | | kinase inhibitor) and Zactima/ZD6474 (a small molecule |
| | | tyrosine kinase inhibitor) and AE-941/Neovastat (a shark |
| | | cartilage component) |
| Angiostatic steroids | Inhibit basement membrane degradation | Includes those disclosed in U.S. Pat. No. 6,011,023 and |
| including but not linitd to | | WO/1990/015816, which are hereby incorporated by |
| tetrahydrocortisol | | reference. |
| Heparin | Inhibit basement membrane degradation | 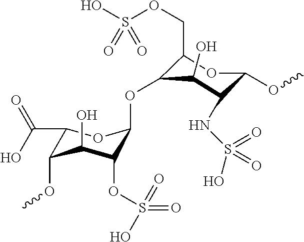 |
| Cartilage-Derived | Inhibit basement membrane degradation | SEQ ID NO 95 (troponin, slow skeletal muscle) |
| Angiogenesis Inhibitors | | SEQ ID NO 96 (troponin, fast skeletal muscle) |
| such as Cartilage-Derived | | SEQ ID NO 97 (troponin, cardiac muscle) |
| Angiogenesis Inhibitory | | SEQ ID NO 98 (chondromodulin-1) |
| factor, troponin and | | |
| chondromodulin | | |
| 2-methoxyestradiol | Inhibit cell proliferation and cell migration and induce apoptosis of endothelial cells | 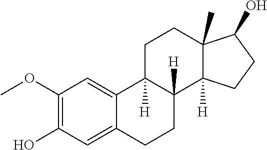 |
| Tecogalan | Inhibit cell proliferation of endothelial cells | A sulfated polysaccharide isolated from various |
| | | Arthrobacter bacterial species. |
| Prolactin | Inhibit bFGF and VEGF | SEQ ID NO 93 |
| α_{V}β₃ inhibitors | Induce apoptosis of endothelial cells | Includes those disclosed in U.S. Pat. No. 6,645,991, |
| | | which is hereby incorporated by reference |
| Linomide | Inhibit cell migration of endothelial cells | LS-2616, quinoline-3-carboxamide |
| Thalidomide (2-(2,6- dioxopiperidin-3-yl) isoindoline-1,3-dione) | Downregulates Angiogenic Genes | 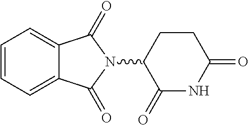 [SMILES: O=C1CCC(N2C(=O)c3ccccc3C2=O)C(=O)N1] |
| C-5013 or lenalidomide | | 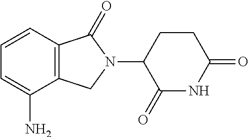 [SMILES: Nc1cccc2c1CN(C1CCC(=O)NC1=O)C2=O] |
| CC-4047 or Actimid, | 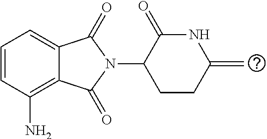 [SMILES: C=C1CCC(N2C(=O)c3cccc(N)c3C2=O)C(=O)N1] | |
| CPS 11 (N-substituted analogue of thalidomide) | 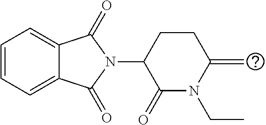 [SMILES: C=C1CCC(N2C(=O)c3ccccc3C2=O)C(=O)N1CC] | |
| CPS 49 (tetraflourinated analogues of thalidomide) | 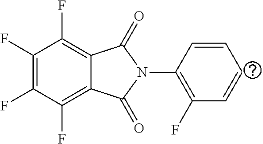 [SMILES: O=C1c2c(F)c(F)c(F)c(F)c2C(=O)N1c1ccccc1F] | |
| CPS 45 (tetraflourinated analogues of thalidomide) | 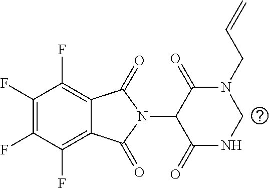 [SMILES: C=CCN1CNC(=O)C(N2C(=O)c3c(F)c(F)c(F)c(F)c3C2=O)C1=O] | |
| EM-12 | | |
| Phthalimidophthalimide | | |
| Cilengitide | Based on the cyclic peptide cyclo(-RGDfV-), which is selective for α_{V} integrines, | 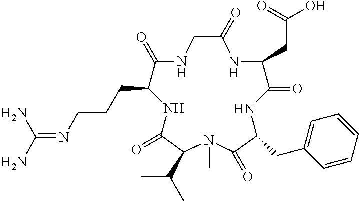 [SMILES: CC(C)[C@H]1C(=O)N[C@@H](CCCN=C(N)N)C(=O)NCC(=O)N[C@@H](CC(=O)O)C(=O)N[C@H](Cc2ccccc2)C(=O)N1C] |
| Combretastatin | Celecoxib phenols. Destabilizes micro-tubules of vascular cells and | 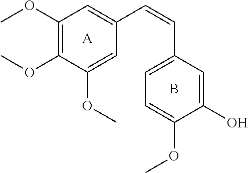 |
| DMXAA (5,6- | Increases NF-kb transcription by phosphory- | A flavonoid analogue |
| dimethylxanthenone-4-acetic | lation leading to the production of proteins | |
| acid) | that change vascular cell shape and organi- | |
| | zation eventually leading to apoptosis of these | |
| | cells | |
| Aplidin/dehydrodidemin B | Induces apoptosis rap- | A marine-derived anti-tumour agent, originally isolated from |
| | idly and persistently, inhibits VEGF secretion | the tunicate Aplidium albicans |
| | and blocks cell-cycle. | |

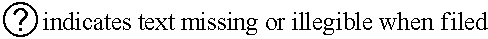

An agent capable of inhibiting angiogenesis and/or an active anti-cancer and/or an anti-metastatic agent according to the present invention may also be small-molecule-receptor tyrosine kinase inhibitors including but not limited to sorafenib, sunitinib, vatalanib, AZD2171, CEP-7055, CHIR258, CP-547632, GW786034, OSI-930, ZK-CDK, AG013736, AMG706, KRN-951, BMS-582664, XL999 or Zactima.

An agent capable of inhibiting angiogenesis and/or an active anti-cancer agent and/or an anti-metastatic agent according to the present invention may also be a compound that inhibits the activity of one or more angiogenesis stimulators including but not limited to FGF, VEGF, VEGFR, NRP-1, Ang1, Tie2, PDGF, PDGFR, TGF-β, endoglin and TGF-β receptors, MCP-1, Integrins α_{V}β₃, α_{V}β₅ and α₅β₁, VE-cadherin, CD31, ephrin, plasminogen activators, plasminogen activator inhibitor-1, NOS, COX-2, such as Celecoxib, AC133, PlGF, MMPs such as Marimastat or Batimasta and Id1/Id3 as listed in table 2. Such agents include but are not limited to antibodies or an antigen-binding fragment thereof, peptide-like antibodies, neutralizing antibodies, antisense-RNA, antisense-DNA or siRNA directed against one or more angiogenesis stimulators including but not limited to FGF, VEGF, VEGFR, NRP-1, Ang1, Tie2, PDGF, PDGFR, TGF-β, endoglin and TGF-β receptors, MCP-1, Integrins α_{V}β₃, α_{V}β₅ and α₅β₁, VE-cadherin, CD31, ephrin, plasminogen activators, plasminogen activator inhibitor-1, NOS, COX-2, AC133 and Id1/Id3 as listed in table 2. The angiogenesis inhibitor according to the present invention may also be an Inhibitor of the notch-DII4 signaling pathway or vascular disrupting agents such as microtubule inhibitors.

TABLE 3
Angiogenetic stimulators and inhibitors thereof

| Stimulator | Mechanism | SEQ ID NO. or structure |
|---|---|---|
| Fibroblast growth factor | Promotes proliferation & | SEQ ID NO 1 (FGF 10) |
| (FGF) | differentiation of endothelial cells, | SEQ ID NO 2 (FGF 11) |
| | smooth muscle cells, and | SEQ ID NO 3 (FGF 12) |
| | fibroblasts | SEQ ID NO 4 (FGF 13) |
| | | SEQ ID NO 5 (FGF 14) |
| | | SEQ ID NO 6 (FGF 16) |
| | | SEQ ID NO 7 (FGF 17) |
| | | SEQ ID NO 8 (FGF 18) |
| | | SEQ ID NO 9 (FGF 19) |
| VEGF | Affects permeability | SEQ ID NO 10 (VEGF A) |
| | | SEQ ID NO 11 (VEGF B) |
| | | SEQ ID NO 12 (VEGF C) |
| | | SEQ ID NO 13 (VEGF D) |
| VEGFR | Integrate survival signals | SEQ ID NO 14 (VEGFR 1) |
| | | SEQ ID NO 15 (VEGFR 2) |
| | | SEQ ID NO 16 (VEGFR 3) |
| Placental growth factor | Growth factor that binds to | SEQ ID NO 12 (VEGF C) |
| (PIGF) or vascular | VEGFR-1 | |
| endothelial growth | | |
| factor-related protein | | |
| Vasohibin | Operates as an intrinsic and highly | SEQ ID NO 94 |
| | specific feedback inhibitor of | |
| | activated endothelial cells engaged | |
| | in the process of angiogenesis | |
| Tumstatin | inhibition of endothelial cell | SEQ ID NO 99 |
| | proliferation and promotion of | |
| | apoptosis | |
| Neuropilin-1 or | Integrates survival signals | SEQ ID NO 17 (Neuropilin-1) |
| Neuropilin-2 (NRP-1 or | | SEQ ID NO 18 (Neuropilin-2) |
| NRP-2) | | |
| Ang1 (Angiopoietin-1) | Stabilize vessels | SEQ ID NO 19 |
| Tie2 (Angiopoietin-1 | Stabilize vessels | SEQ ID NO 20 |
| receptor) | | |
| PDGF (BB-homodimer) | Recruit smooth muscle cells | SEQ ID NO 21 |
| Platelet-derived growth | | |
| factor B chain | | |
| PDGFR platelet- | Recruit smooth muscle cells | SEQ ID NO 22 (Alpha-type platelet-derived growth factor receptor) |
| derived growth factor | | SEQ ID NO 23 (Beta-type platelet-derived growth factor receptor) |
| receptor | | SEQ ID NO 24: (Platelet-derived growth factor receptor-like protein) |
| Transforming growth | Increase extracellular matrix | SEQ ID NO 25 (TGF beta-1) |
| factor-β (TGF-β) | production | SEQ ID NO 26 (TGF beta-2) |
| | | SEQ ID NO 27 (TGF beta-3) |
| TGF-β receptors | Increase extracellular matrix | SEQ ID NO 28 (TGF beta receptor type III) |
| | production | |

| | | |
|---|---|---|
| endoglin | Increase extracellular matrix | SEQ ID NO 29 |
| | production | |
| Integrins α_{V}β₃ | Bind matrix macromolecules and | SEQ ID NO 30 (Integrin alpha-5) |
| | proteinases | SEQ ID NO 31 (Integrin beta-3) |
| Integrins α_{V}β₅ | Bind matrix macromolecules and | SEQ ID NO 30 (Integrin alpha-5) |
| | proteinases | SEQ ID NO 32 (Integrin beta-5) |
| Integrins α₅β₁ | Bind matrix macromolecules and | SEQ ID NO 30 (Integrin alpha-5) |
| | proteinases | SEQ ID NO 33 (Integrin beta-1) |
| Integrin antagonists such as cilengitide | α_{V}β₃ antagonist | 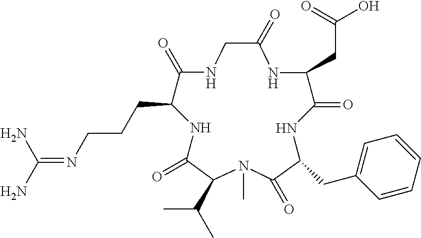 [SMILES: CC(C)[C@H]1C(=O)N[C@@H](CCCN=C(N)N)C(=O)NCC(=O)N[C@@H](CC(=O)O)C(=O)N[C@H](Cc2ccccc2)C(=O)N1C] |
| Antibodies to integrins | Antiobodies to α_{V}β₃ | |
| including but not limited | | |
| to LM-609; Vitaxin 2 | | |
| CD31 | endothelial junctional molecules | SEQ ID NO 34 |
| VE-cadherin | endothelial junctional molecules | SEQ ID NO 35 |
| Ephrin | Determine formation of arteries or | SEQ ID NO 36 (Ephrin-A1) |
| | veins | SEQ ID NO 37 (Ephrin-A2) |
| | | SEQ ID NO 38 (Ephrin-A3) |
| | | SEQ ID NO 39 (Ephrin-A4) |
| | | SEQ ID NO 40 (Ephrin-A5) |
| | | SEQ ID NO 41 (Ephrin-B1) |
| | | SEQ ID NO 42 (Ephrin-B2) |
| | | SEQ ID NO 43 (Ephrin-B3) |
| Plasminogen activators | Remodels extracellular matrix, | SEQ ID NO 44 |
| such as for example | releases and activates growth | |
| Tissue-type | factors | |
| plasminogen activator | | |
| Plasminogen activator | Stabilizes nearby vessels | SEQ ID NO 45 |
| inhibitor-1 | | |
| NOS (nitric oxide | | SEQ ID NO 100 (brain variant) |
| synthase) | | SEQ ID NO 101 (inducible) |
| | | SEQ ID NO 102 (IIB fragment) |
| | | SEQ ID NO 103 (IIC fragment) |
| | | SEQ ID NO 104 (endothelial) |
| COX-2 (Prostaglandin | | SEQ ID NO 46 |
| G/H synthase 2) | | |
| Celecoxib (a COX-2 inhibitor) | A non-steroidal anti-inflammatory drug (NSAID). Decreases vascular permeability, endothelial cell proliferation and migration, MMP production and affects integrin signalling pathway | 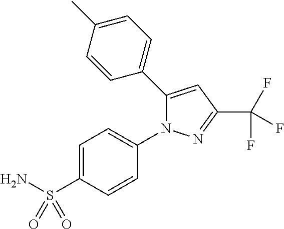 [SMILES: Cc1ccc(-c2cc(C(F)(F)F)nn2-c2ccc(S(N)(=O)=O)cc2)cc1] |
| AC133 antigen | Regulates angioblast differentiation | |
| DNA-binding protein | Regulates endothalial | SEQ ID NO 47 |
| inhibitor ID-1 | transdifferentiation | |
| DNA-binding protein | Regulates endothalial | SEQ ID NO 48 |
| inhibitor ID-3 | transdifferentiation | |
| Matrix | Inhibit basement membrane | Synthetic inhibitors generally contain a chelating group which binds the |
| metalloproteinase | degradation | catalytic zinc atom at the MMP active site tightly. Common chelating |
| (MMP) inhibitors | | groups include hydroxamates, carboxylates, thiols, and phosphinyls. |
| | | Including but not limited to see below row: |
| Marimastat (an MMP inhibitor) | | 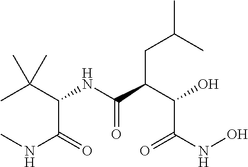 [SMILES: CNC(=O)[C@@H](NC(=O)[C@@H](CC(C)C)[C@H](O)C(=O)NO)C(C)(C)C] |
| Batimastat (an MMP inhibitor) | | 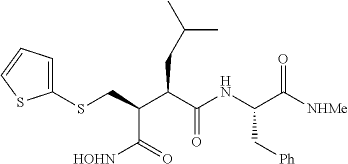 |

The pulmonary administration of an anti-angiogenic agent according to the present invention may be combined with the administration of active anti-cancer agents chemotherapeutics either systemically or local pulmonary deposition. The active anti-cancer agents chemotherapeutics may be any of the anti-angiogenic agents mentioned herein above or any chemotherapeutic drug, cytotoxic agent or a PDGFR inhibitor.

Thus the pulmonary administration of an anti-angiogenic agent according to the present invention may be combined with the systemic administration of active anti-cancer agents or chemotherapeutics including but not limited to:
Alkylating agents, such as Cisplatin, carboplatin, oxaliplatin, mechlorethamine, cyclophosphamide and chlorambucil.
Anti-metabolites, such as purine analogues, pyrimidine analogues and antifolates.
Plant alkaloids and terpenoids. The compounds includes but are not limited to taxanes including paclitaxel/taxol, docetaxel, and vinca alkaloids including Vincristine, Vinblastine, Vinorelbine, Vindesine and Podophyllotoxin which is used to produce two other cytostatic drugs, etoposide and teniposide.
Topoisomerase inhibitors, including the type I topoisomerase inhibitors camptothecins: irinotecan and topotecan and type II inhibitors such as amsacrine, etoposide, etoposide phosphate, and teniposide.
Antitumour antibiotics, such as dactinomycin, actinomycin, anthracyclines including doxorubicin, daunorubicin and epirubicin and other cytotoxic antibiotics such as bleomycin, plicamycin and mitomycin.
Monoclonal antibodies such as trastuzumab (Herceptin), cetuximab, and rituximab (Rituxan or Mabthera) and Bevacizumab (Avastin)

### Functional Homologues of Polypeptides

A functional homologue of a polypeptide of a given sequence within the present invention is a polypeptide sharing at least some sequence identity with the given sequences and which shares at least one function, preferably, having anti-angiogenic, anti-cancer and/or anti-metastatic function.

Preferably, evolutionary conservation between polypeptides of different closely related species, e.g. assessed by sequence alignment, can be used to pinpoint the degree of evolutionary pressure on individual residues. Preferably, polypeptide sequences from at least 2, preferably at least 3, more preferably at least four different species where the function of the polypeptide is conserved are compared, for example but not limited to mammals including rodents, monkeys and apes. Conserved residues are more likely to represent essential amino acids that cannot easily be substituted as compared to residues that change between species. For example, such an alignment may be performed using ClustalW from EBML-EBI. It is evident from the above that a reasonable number of modifications or alterations of a polypeptide sequence does not interfere with the activity of a given polypeptide. Thus, preferably, functional homologues of a given polypeptide comprise all residues, which are conserved between at least 4, such as at least 3, for example at least 2 different species. Functional homologues may thus comprise one or more amino acid substitutions at residues, which are not conserved between at least 4, such as at least 3, for example at least 2 different species.

It is preferred that at least some, for example at least 50%, such as all amino acid substitutions are “conservative”. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined group exhibit similar or substantially similar characteristics, preferably the groups are the groups listed below in “Lower levels of similarity”, even more preferably the groups are the groups listed below in “High level of similarity”.

Within the meaning of the term “conservative amino acid substitution” as applied herein, one amino acid may be substituted for another within the groups of amino acids indicated herein below:

### Lower Levels of Similarity:

### Polarity:

i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, Ile, Phe, Trp, Pro, and Met)
   Hydrophilic or hydrophobic:
iii) Hydrophobic amino acids (Ala, Cys, Gly, Ile, Leu, Met, Phe, Pro, Trp, Tyr, Val)
iv) Hydrophilic amino acids (Arg, Ser, Thr, Asn, Asp, Gln, Glu, His, Lys)

### Charges:

v) Neutral amino acids (Ala, Asn, Cys, Gln, Gly, Ile, Leu, Met, Phe, Pro, Ser, Thr, Trp, Tyr, Val)
vi) Basic amino acids (Arg, His, Lys)
vii) Acidic amino acids ((asp, Glu)

### High Level of Similarity:

viii) Acidic amino acids and their amides (Gln, Asn, Glu, Asp)
ix) Amino acids having aliphatic side chains (Gly, Ala Val, Leu, Ile)
x) Amino acids having aromatic side chains (Phe, Tyr, Trp)
xi) Amino acids having basic side chains (Lys, Arg, His)
xii) Amino acids having hydroxy side chains (Ser, Thr)
xiii) Amino acids having sulphor-containing side chains (Cys, Met),

More preferred conservative amino acids substitution groups are: valine-leucine-isoleucine, phenylalanine-tyrosine, lysine-arginine, alanine-valine, and asparagine-glutamine.

It is clear from the above outline that the same functional homologue or fragment thereof may comprise more than one conservative amino acid substitution from more than one group of conservative amino acids as defined herein above.

Polypeptides of the invention may comprise standard and non-standard amino acids or mixtures of both. It is preferred that the polypeptides only comprise standard amino acids. There are twenty standard naturally occurring amino acids and two special amino acids, selenocysteine and pyrrolysine, as well as a vast number of “nonstandard amino acids” which are not incorporated into protein in vivo. Examples of nonstandard amino acids include the sulfur-containing taurine and the neurotransmitters GABA and dopamine. Other examples are lanthionine, 2-Aminoisobutyric acid, and dehydroalanine. Further non standard amino acids are ornithine and citrulline.

Non-standard amino acids are usually formed through modifications to standard amino acids. For example, taurine can be formed by the decarboxylation of cysteine, while dopamine is synthesized from tyrosine and hydroxyproline is made by a posttranslational modification of proline (common in collagen). Examples of non-naturally occurring amino acids are those listed e.g. in 37 C.F.R. section 1.822(b)(4), all of which are incorporated herein by reference.

Both standard and non-standard amino acid residues described herein can be in the “D” or “L” isomeric form, preferably “L” isomeric form.

It is contemplated that a functional equivalent according to the invention may comprise any amino acid including non-standard amino acids. In preferred embodiments a functional equivalent comprises only standard amino acids.

The standard and/or non-standard amino acids may be linked by peptide bonds or by non-peptide bonds, preferably however by peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

A functional homologue within the scope of the present invention is a polypeptide that exhibits at least some sequence identity with a polypeptide of a given sequence, preferably functional homologues have at least 75% sequence identity, for example at least 80% sequence identity, such as at least 85% sequence identity, for example at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with a given polypeptide sequence.

Sequence identity can be calculated using a number of well-known algorithms and applying a number of different gap penalties. The sequence identity is calculated relative to the full-length sequence of the reference polypeptide. Any sequence alignment tool, such as but not limited to FASTA, BLAST, or LALIGN may be used for searching homologues and calculating sequence identity. Moreover, when appropriate any commonly known substitution matrix, such as but not limited to PAM, BLOSSUM or PSSM matrices may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Functional homologues may in one embodiment further comprise chemical modifications such as ubiquitination, labeling (e.g., with radionucleotides, various enzymes, etc.), pegylation (derivatization with polyethylene glycol), or by insertion (or substitution by chemical synthesis) of amino acids such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same molecules, including dimers or unrelated chemical moieties. Such functional equivalents are prepared by linkage of functionalities to groups which are found in fragment including at any one or both of the N- and C-termini, by means known in the art. Functional homologues may also be deletion or addition mutants. The addition may be addition of at least one amino acid, an addition of from preferably 2 to 250 amino acids, such as from 10 to 20 amino acids, for example from 20 to 30 amino acids, such as from 40 to 50 amino acids.

A functional homologue may be a deletion mutant which have at least 75% sequence identity, for example at least 80% sequence identity, such as at least 85% sequence identity, for example at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity.

Deletion mutants suitably comprise at least 20 or 40 consecutive amino acid and more preferably at least 80 or 100 consecutive amino acids in length.

It is preferred that functional homologues of a given polypeptide comprises at the most 500, more preferably at the most 400, even more preferably at the most 300, yet more preferably at the most 200, such as at the most 175, for example at the most 160, such as at the most 150 amino acids in addition to the sequence of the given polypeptide.

A functional homologue of an agent capable of inhibiting angiogenesis and/or an active anti-cancer and/or an anti-metastatic agent may be a polypeptide having at least 70% sequence identity with SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 51, 53, 54, 55, 56, 57, 58, 59, 60, 61, 62, 63, 63, 65, 66, 67, 68, 69, 70, 71, 72, 72, 73, 74, 75, 76, 77, 78, 79, 80, 81, 82, 83, 84, 85, 86, 87, 88, 89, 90, 91, 92, 93, 94, 95, 96, 97 or 98 and having anti-angiogenic, anti-cancer and/or anti-metastatic function.

Methods for assaying anti-angiogenic function include but are not limited to those described in Auerbach et al., (2003) and Walter et al., (2004), both of which are hereby incorporated by reference.

Methods for assaying anti-metastatic function include but are not limited to the lung tumor colonization assay (Fidler, I. J., Nature (London) (New Biol) 242:148 (1973)), Colorimetric heparinase assay for alternative anti-metastatic activity (S. C. Ahn, B. Y. Kim, W. K. Oh, Y. M. Park, H. M. Kim and J. S. Ahn, Life Sciences, Volume 79, Issue 17, 20 Sep. 2006, Pages 1661-1665) and those described in U.S. Pat. No. 5,039,662, all of which are hereby incorporated by reference.

Other methods for evaluating the efficacy of an angiogenesis inhibitor includes: taking biopsies to analyse protein expression as a marker, microvascular density, presivascular cell coverage of tumor vessels or cell proliferation/apoptosis genomic analysis;
Measuring the tumor interstitial fluid pressure;
Measuring of tissue oxygenation;
Measuring wound healing time;
Measuring the concentration of viable circulating endothelial cells (CECs) in the blood;
Measuring the concentration of viable circulating progenitor cells (CPCs) in the blood;
Measuring the protein level in plasma of proteins such as VEGF, PlGF, TSP1 and adhesion molecules;
Measuring the protein level in ascites;
Measuring the protein level inpleural effusions;
CT imaging of blood flow and volume, permeability-surface area product mean product transit time;
PET imaging of tracer uptake;
MRI of blood flow and permeability;
Measuring the protein level in urine

### (Jain R K, Duda D G, Clark J W, Loefller J S (2006) Nature Clinical Practice, Vol. 3 No. 1)

As used herein the expression “variant” refers to polypeptides or proteins which are homologous to the basic protein, which is suitably an agent capable of inhibiting angiogenesis and/or an active anti-cancer and/or an anti-metastatic agent including but not limited to those defined by SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 51, 53, 54, 55, 56, 57, 58, 59, 60, 61, 62, 63, 63, 65, 66, 67, 68, 69, 70, 71, 72, 72, 73, 74, 75, 76, 77, 78, 79, 80, 81, 82, 83, 84, 85, 86, 87, 88, 89, 90, 91, 92, 93, 94, 95, 96, 97 or 98 but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

### Preparation of Polypeptides

Polypeptides may be purified from natural sources, which should be selected according to the occurrence of the polypeptide. Non-limiting examples of natural sources includes cell extracts, tissue extracts, plant extracts, body fluids, such as saliva or serum, milk or eggs. Polypeptides may also be recombinantly produced as described in more details herein below and then optionally be purified from host cells expressing the heterologous protein, from host organisms, such as transgenic plants or animals expressing the heterologous polypeptide or from tissue culture medium from host cells expressing the heterologous polypeptides.

Purification of proteins in general involves one or more steps of removal of or separation from contaminating nucleic acids, phages and/or viruses, other proteins and/or other biological macromolecules. The procedure may comprise one or more protein isolation steps. Any suitable protein isolation step may be used with the present invention. The skilled person will in general readily be able to identify useful protein isolation steps for a given polypeptide using routine experimentation.

The protein isolation steps useful with the present invention may be commonly used methods for protein purification including for example chromatographic methods such as for example gas chromatography, liquid chromatography, ion exchange chromatography and/or affinity chromatography; filtration methods such as for example gel filtration and ultrafiltration; precipitation, such as ammonium sulphate precipitation and/or gradient separation such as sucrose gradient separation. The purification may comprise one or more of the aforementioned methods in any combination.

The aforementioned methods are well known to the skilled person and may for example be performed as described in the “Protein Separation Handbook Collection” including the titles “Antibody Purification”, “The Recombinant Protein Handbook”, “Protein Purification”, “Ion Exchange Chromatography”, “Affinity Chromatography”, “Hydrophobic Interaction Chromatography”, “Gel Filtration”, “Reversed Phase Chromatography”, “Expanded Bed Adsorption” and “Chromatofocusing” prepared by Amersham Biosciences and available from GE.

The polypeptides of the invention may also be recombinantly prepared, in particular functional homologues are preferably produced recombinantly. Useful recombinant production methods includes conventional methods known in the art, such as by expression of heterologuos polypeptide or functional homologues thereof in suitable host cells such as E. coli, S. cerevisiae or S. pombe or insect or mammalian cells suitable for production of recombinant proteins (see below). The skilled person will in general readily be able to identify useful recombinant techniques for the production of recombinant proteins in general.

In one embodiment the polypeptides are produced in a transgene plant or animal. By a transgenic plant or animal in this context is meant a plant or animal which has been genetically modified to contain and express a nucleic acid encoding the given polypeptide or functional homolgue hereof.

In one aspect of the present invention, the polypeptide is produced by host cells comprising a first nucleic acid sequence encoding the given polypeptide or a functional homologue thereof operably associated with a second nucleic acid capable of directing expression in said host cells. The second nucleic acid sequence may thus comprise or even consist of a promoter that will direct the expression of protein of interest in said cells. A skilled person will be readily capable of identifying useful second nucleic acid sequence for use in a given host cell.

The process of producing recombinant polypeptide or a functional homologue thereof in general comprises the steps of:
providing a host cell
preparing a gene expression construct comprising a first nucleic acid sequence encoding a given polypeptide or a functional homologue thereof operably linked to a second nucleic acid sequence capable of directing expression of said protein of interest in the host cell
transforming the host cell with the construct,
cultivating the host cell, thereby obtaining expression of the polypeptide or the functional homologue thereof.

The recombinant polypeptide thus produced may be isolated by any conventional method for example by any of the protein purification methods described herein above. The skilled person will be able to identify a suitable protein isolation steps for purifying any protein of interest.

In a preferred embodiment of the invention, the polypeptide is recombinantly produced in vitro in host cells and is isolated from cell lysate, cell extract or from tissue culture supernatant. In a more preferred embodiment polypeptide is produced by host cells that are modified in such a way that they express the polypeptide of interest. In an even more preferred embodiment of the invention said host cells are transformed to produce and excrete the polypeptide.

The gene expression construct may comprise a viral based vector, such as a DNA viral based vector, a RNA viral based vector, or a chimeric viral based vector. Examples of DNA viruses are cytomegalo virus, Herpex Simplex, Epstein-Barr virus, Simian virus 40, Bovine papillomavirus, Adeno-associated virus, Adenovirus, Vaccinia virus, and Baculo virus. However, the gene expression construct may for example only comprise a plasmid based vector.

In one aspect the invention provides an expression construct encoding a given polypeptide or functional homologues thereof, featured by comprising one or more intron sequences for example from the native gene. Additionally, it may contain a promoter region derived from a viral gene or a eukaryotic gene, including mammalian and insect genes.

The promoter region is preferably selected to be different from the native promoter, and preferably in order to optimize the yield, the promoter region is selected to function most optimally with the vector and host cells in question.

In a preferred embodiment, the promoter region is selected from a group comprising Rous sarcoma virus long terminal repeat promoter, and cytomegalovirus immediate-early promoter, and elongation factor-1 alpha promoter.

In another embodiment the promoter region is derived from a gene of a microorganism, such as other viruses, yeasts and bacteria.

In order to obtain a greater yield of recombinant LA or functional homologue thereof, the promoter region may comprise enhancer elements, such as the QBI SP163 element of the 5′ end untranslated region of the mouse vascular endothelian growth factor gene

One process for producing a recombinant polypeptide having anti-angiogenic, anti-cancer and/or anti-metastatic function according to the invention is characterised in that the host cell culture is may be eukaryotic, for example a mammalian cell culture or a yeast cell culture.

Useful mammalian cells may for example be human embryonal kidney cells (HEK cells), such as the cell lines deposited at the American Type Culture Collection with the numbers CRL-1573 and CRL-10852, chick embryo fibroblast, hamster ovary cells, baby hamster kidney cells, human cervical carcinoma cells, human melanoma cells, human kidney cells, human umbilical vascular endothelium cells, human brain endothelium cells, human oral cavity tumor cells, monkey kidney cells, mouse fibroblast, mouse kidney cells, mouse connective tissue cells, mouse oligodendritic cells, mouse macrophage, mouse fibroblast, mouse neuroblastoma cells, mouse pre-B cell, mouse B lymphoma cells, mouse plasmacytoma cells, mouse teratocarcinoma cells, rat astrocytoma cells, rat mammary epithelium cells, COS, CHO, BHK, 293, VERO, HeLa, MDCK, WI38, and NIH 3T3 cells.

The host cells may also either be prokaryotic cells or yeast cells. Prokaryotic cells may for example be E. coli. Yeast cells may for example be Saccharomyces, Pichia or Hansenula.

The aforementioned methods are well known to the skilled person and may for example be performed as described in the Current Protocols in Molecular Biology, 2001, by John Wiley and Sons, Inc. edited by Frederick M. Ausubel et al.

For use in the present invention, a variety of angiogenesis inhibitor polypeptides can also be readily designed and manufactured utilizing recombinant DNA techniques well known to those skilled in the art. For example, the amino acid sequence can vary from the naturally occurring sequences at the primary structure level by amino acid substitutions, insertions, deletions, and the like. In general, modifications of the genes are readily accomplished by a variety of well-known techniques, such as site-directed mutagenesis (see, Gillman and Smith, Gene 8:81-97 (1979) and Roberts, S. et al., Nature 328:731-734 (1987) and U.S. Pat. No. 5,032,676, all of which are incorporated herein by reference). Most modifications are evaluated by screening in a suitable assay for the desired characteristic.

### Pulmonary Administration

When used herein the term “deposition of drugs in the lungs” and other variations on this theme is meant to describe local pulmonary drug administration and not systemic drug administration via the airways with the purpose of minimising the systemic spill over of the anti-angiogenetic drug in order to avoid or minimise the many adverse effects connected with systemic administration of anti-angiogentic agents.

As the airways of the lung become smaller and more highly branched, they become increasingly difficult to penetrate with aerosol. To optimize deposition in either the oropharyngeal/tracheobronchial region for a local dose or the bronchial/pulmonary region for a systemic dose, a key parameter to control is particle size. It is generally accepted that for particles with aerodynamic diameters less than 5 microns, the greatest fractional deposition is in the bronchial/pulmonary region of the lung. Deposition in the oropharyngeal/tracheobronchial region occurs with particles ranging from approximately 5 to 100 μm.

For inhaled formulations, the balance between desired local effects and undesired systemic activity can be expressed by L/T, where L represents bioavailability of drug from the lungs and T represents total systemic bioavailability. A high L/T is desirable as this implies efficient drug delivery to the target site, and minimization of unwanted activity from non-targeted drug delivery.

A schematic representation of the difference between systemic drug administration, systemic pulmonary drug administration and local pulmonary drug deposition can be seen in FIG. 1.

Methods of intratracheal, intrabronchial, intraalveolar or bronchio-alveolar administration include, but are not limited to, spraying, lavage, inhalation, flushing or instillation, using as fluid a physiologically acceptable composition in which the angiogenesis inhibitor have been dissolved or as a dry powder inhalation. When used herein the terms “intratracheal, intrabronchial or intraalveolar or bronchio-alveolar administration” include all forms of such administration whereby one or more agents capable of inhibiting angiogenesis and/or active anti-cancer agents and/or anti-metastatic agents are applied into the trachea, the bronchi or the alveoli, respectively, whether by the instillation of a solution of the angiogenesis inhibitor, by applying the angiogenesis inhibitor in a powder form, or by allowing one or more agents capable of inhibiting angiogenesis and/or active anti-cancer agents and/or anti-metastatic agents to reach the relevant part of the airway by inhalation of one or more agents capable of inhibiting angiogenesis and/or active anti-cancer agents and/or anti-metastatic agents as an aerosolized or nebulized solution or suspension or inhaled powder or gel, with or without added stabilizers or other excipients.

Methods of intrabronchial/alveolar administration include, but are not limited to, bronchoalveolar lavage (BAL) according to methods well known to those skilled in the art, using as a lavage fluid a physiologically acceptable composition in which the angiogenesis inhibitor been dissolved or indeed by any other effective form of intrabronchial administration including the use of inhaled powders containing the angiogenesis inhibitor in dry form, with or without excipients, or the direct application of one or more agents capable of inhibiting angiogenesis and/or active anti-cancer agents and/or anti-metastatic agents itor, in solution or suspension or powder form during bronchoscopy. Methods for intratracheal administration include, but are not limited to, blind tracheal washing with a similar solution of dissolved agent capable of inhibiting angiogenesis and/or active anti-cancer agents and/or anti-metastatic agents or a suspension of one or more agents capable of inhibiting angiogenesis and/or active anti-cancer agents and/or anti-metastatic agents, or the inhalation of nebulized fluid droplets containing dissolved agent capable of inhibiting angiogenesis and/or active anti-cancer agents and/or anti-metastatic agents a suspension of one or more agents capable of inhibiting angiogenesis and/or active anti-cancer agents and/or anti-metastatic agents, obtained by use of any nebulizing apparatus adequate for this purpose.

In another embodiment, intratracheal, intrabronchial or intraalveolar administration does not include inhalation of the angiogenesis inhibitor but the instillation or application of a solution of one or more agents capable of inhibiting angiogenesis and/or active anti-cancer agents and/or anti-metastatic agents or a powder or a gel containing one or more agents capable of inhibiting angiogenesis and/or active anti-cancer agents and/or anti-metastatic agents into the trachea or lower airways.

Other preferred methods of administration may include using the following devices:
1. Pressurized nebulizers using compressed air/oxygen mixture
2. Ultrasonic nebulizers
3. Electronic micropump nebulizers (e.g. Aeroneb Professional Nebulizer)
4. Metered dose inhaler (MDI)
5. Dry powder inhaler systems (DPI),
6. Unit dose inhalant

The present invention provides a useful new addition to the methods of treating metastases. Furthermore, the administration of one or more agents capable of inhibiting angiogenesis and/or active anti-cancer agents and/or anti-metastatic agents via the airway is expected to avoid the spill-over of the agents to systemic circulation. At the same time, the application of the agents via the airway is expected to potentiate their effect on inhibition of angiogenesis and/or pulmonary metastasis when compared with their systemic administration. It is expected that the total dosage of an angiogenesis inhibitor may be used alone locally within the airspaces or may be divided between the conventional intravenous route and the airway route of the present invention to obtain the optimal balance between the systemic and local pulmonary effects of the treatment, and a reduced incidence of drug adverse effect. Furthermore, the time interval (“window of opportunity”) during which the intravenous use of one or more agents capable of inhibiting angiogenesis and/or active anti-cancer agents and/or anti-metastatic agents is likely to be beneficial is limited. A longer time interval of drug response may be expected when the agent is used even in the late metastatic phase.

The aerosol may be delivered by via a) facemasks or b) via endotracheal tubes in intubated patients during mechanical ventilation (device 1, 2 and 3). The devices 4, 5 and 6 can also be used by the patient without assistance provided that the patient is able to self-activate the aerosol device.

Thus, in one embodiment the effective amount of one or more agents capable of inhibiting angiogenesis and/or active anti-cancer agents and/or anti-metastatic agents or a functional homologue thereof is administered by intratracheal, intrabronchial, intraalveolar or bronchio-alveolar administration.

In another embodiment the subject is administered a solution of one or more agents capable of inhibiting angiogenesis and/or active anti-cancer agents and/or anti-metastatic agents thereof via bronchoalveolar lavage.

In another embodiment the subject is administered a solution of one or more agents capable of inhibiting angiogenesis and/or active anti-cancer agents and/or anti-metastatic agents via blind tracheal washing alone as the sole intervention or in combination with systemic administration of chemotherapeutic anticancer therapy.

In another embodiment the subject is administered a nebulized solution or a suspension of one or more agents capable of inhibiting angiogenesis and/or active anti-cancer agents and/or anti-metastatic agents.

In another embodiment the subject is administered a nebulized aerosol or inhaled powder form of one or more agents capable of inhibiting angiogenesis and/or active anti-cancer agents and/or anti-metastatic agents.

In another embodiment the subject is administered a pegylated, liposomal or nanoparticle prepared form of one or more agents capable of inhibiting angiogenesis and/or active anti-cancer agents and/or anti-metastatic agents.

In another embodiment the subject is administered one or more agents capable of inhibiting angiogenesis and/or active anti-cancer agents and/or anti-metastatic agents by direct application during bronchoscopy.

In another embodiment the subject is a mammal.

In another embodiment the mammal is a human.

In another embodiment the human is a child younger than 12 years of age.

In another embodiment the human is an adult older than 12 years of age.

Preferred concentrations for a solution comprising one or more agents capable of inhibiting angiogenesis and/or active anti-cancer agents and/or anti-metastatic agents are in the range of 0.1 μg to 10000 μg active ingredients per ml solution. The suitable concentrations are often in the range of from 0.1 μg to 5000 μg per ml solution, such as in the range of from about 0.1 μg to 3000 μg per ml solution, and especially in the range of from about 0.1 μg to 1000 μg per ml solution, such as in the range of from about 0.1 μg to 250 μg per ml solution. A preferred concentration would be from about 0.1 to about 5.0 mg, preferably from about 0.3 mg to about 3.0 mg, such as from about 0.5 to about 1.5 mg and especially in the range from 0.8 to 1.0 mg per ml solution. A preferred concentration would be from about 0.1 to about 5.0 mg, preferably from about 0.3 mg to about 3.0 mg, such as from about 0.2 to about 2.5 mg and especially in the range from 0.2 to 1.0 mg per ml solution. Typical systemic doses are:
Lenalidomide, administered orally at the dose of 25 mg q.d. for 21 days.
Thalidomide (n=129; 100 mg per day continuously until any sign of relapse or progressive disease.
Suramin dose initially for the first cycle was 240 mg/m(2)

### Pharmaceutical Compositions

Pharmaceutical compositions or formulations for use in the present invention include a preparation of one or more agents capable of inhibiting angiogenesis and/or active anti-cancer agents and/or anti-metastatic agents in combination with, preferably dissolved in, a pharmaceutically acceptable carrier, preferably an aqueous carrier or diluent. The pharmaceutical composition may be a solid, a liquid, a gel or an aerosol. A variety of aqueous carriers may be used, such as 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration.

In one embodiment a freeze-dried angiogenesis inhibitor preparation may be pre-packaged for example in single dose units. In an even more preferred embodiment the single dose unit is adjusted to the patient.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc. The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some embodiments the micellar formulation may be mixed with propellants such as tetrafluoroethane, heptafluoroethane, dimethylfluoropropane, tetrafluoropropane, butane, isobutane, dimethyl ether and other non-CFC and CFC propellants, especially when delivered (e.g. applied to the buccal mucosa) through aerosol devices, e.g. metered dose inhalers (MDIs).

In some cases, it will be advantageous to include a compound, which promotes delivery of the active substance to its target.

In some embodiments the compositions of the present invention are formulated as aerosols. In order to efficiently reach the lung, the formulation maybe atomized into particles having aerodynamic sizes between approximately 1 and 10, preferably between 2 and 5 micrometers Such aerosols may generally comprise one or more agents capable of inhibiting angiogenesis, one or more propellants and either a surfactant or a solvent.

Thus in certain embodiments the compositions according to the present invention may comprise a propellant including but not limited to fluorocarbons and hydrogen-containing chlorofluorocarbons, and a number of medicinal aerosol formulations using such propellant systems are disclosed in, for example, EP 0372777, WO91/04011, WO91/11173, WO91/11495 and WO91/14422.

Suitable solvents for the pharmaceutical preparation within the scope of the present inventions are solutions containing at least 70% (v/v) of ethanol; solutions containing at least 85% (v/v) are preferred whilst solutions having an ethanolcontent of more than 95% (v/v) are particularly preferred. The concentration is given in percent by volume (v/v), the remainder being water. Most particularly preferred is ethanol which already contains small amounts of water, e.g. 96% ethanol, so that it is no longer hygroscopic and evaporates azeotropically.

One alternative is the development of nebulizers in which aqueous solutions of pharmacologically-active substances are sprayed under high pressure so as to produce a mist of inhalable particles. The advantage of these nebulisers is that there is no need to use any propellant gases whatsoever. Solutions of defined volumes containing active substances are sprayed, using high pressures through small nozzles so as to produce inhalable aerosols with a preferred particle size of between 1 and 10, preferably between 2 and 5 micrometers.

### Dosing Regimes

According to the present invention, a pharmaceutically effective amount or a therapeutically effective amount is to be understood as an amount sufficient to induce a desired biological result. The result can be inhibition of the formation of new blood vessels.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 10 mg per kilo body weight.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 10000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. Administration may be performed once or may be followed by subsequent administrations. 0.1 μg to 5000 μg per kilo body weight, such as in the range of from about 0.1 μg to 3000 μg per kilo body weight, and especially in the range of from about 0.1 μg to 1000 μg per kilo body weight, preferably in the range of 5 μg to 500 μg, even more about 50 μg to about 200 μg administered via inhalation once or twice daily. The dosage will vary with the age, sex and weight of the subject to be treated. A preferred dosage of multimeric forms would be in the interval 1 mg to 70 mg per 70 kg body weight.

Suitable daily dosage ranges are per kilo body weight per day normally of the order of several hundred μg active ingredient per day with a preferred range of from about 0.1 μg to 10000 μg per kilo body weight per day. Using monomeric forms of the compounds, the suitable dosages are often in the range of from 0.1 μg to 5000 μg per kilo body weight per day, such as in the range of from about 0.1 μg to 3000 μg per kilo body weight per day, and especially in the range of from about 0.1 μg to 1000 μg per kilo body weight per day, when based on monomeric forms having a sequence identical to SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 51, 53, 54, 55, 56, 57, 58, 59, 60, 61, 62, 63, 63, 65, 66, 67, 68, 69, 70, 71, 72, 72, 73, 74, 75, 76, 77, 78, 79, 80, 81, 82, 83, 84, 85, 86, 87, 88, 89, 90, 91, 92, 93, 94, 95, 96, 97 or 98 for functional homologues and fragments the dose is calculated based on the molecular weight of the monomeric form to the molecular weight of the homologues or fragments.

Duration of dosing will typically range from 1 day to about 4 months, such as 2 days to about 3 months, for example in the range of 1-2 days to 2 months, such as in the range of 1-2 days to 1 month.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging comprising from 1 to 7 dosage units, thereby having dosage units for one or more days, or from 7 to 21 dosage units, or multiples thereof, thereby having dosage units for one week of administration or several weeks of administration.

The dosage units can be as defined above. The medical packaging may be in any suitable form for intratracheal, intrabronchial, bronchioalveolar or intraalveolar administration. In a preferred embodiment the packaging is in the form of a vial, ampule, tube, blister pack, cartridge or capsule.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

### EXAMPLES

### Example 1

### Protocol for Local Pulmonary Treatment with Thalidomide Through Bronchoalveolar Lavage (BAL)

### I. Patient Group to be Treated:

Patients with cancer where the cancer has metastasized to the lungs.

### II. Treatment Regime:

Local administration of 3×20 mg thalidomide dissolved in 20 ml of normal saline via Bronchoalveolar lavage (BAL).

### III. Analysis of Results:

a) Monitoring of a series of chest x-ray and/or thoracic CT-scan combined with signs and symptoms of pulmonary dysfunction, .e.g dyspnea at rest or after exercise, reduced pulmonary function using forced expiratory flow in first second (FEV1) and measurements of vital capacity (VC); oxygenation capacity as by monitoring the PaO₂/FiO₂ ratio (arterial oxygen tension in mmHg over inspired oxygen fraction).

A successful treatment results in disappearance or reduction in size of metastases, in radiography of the lung fields before and after treatment and/or on thoracic CT-scan, and/or reduced signs and symptoms of pulmonary dysfunction i.e. disappearance of dyspnea, increased FEV1 and VC, eventually combined with an improved SAT O₂ measured using pulse oximetry, provided that the first SAT O₂% was abnormally low

### Example 2

### Protocol for Local Pulmonary Treatment with Thalidomide Through Inhalation

### I. Patient Group to be Treated:

Patients with cancer where the cancer has metastasized to the lungs.

### II. Treatment Regime:

Local administration of 3×20 mg thalidomide via a nebulizer (Aeroneb®).

### III. Analysis of Results:

A successful treatment results in 1) disappearance or reduction in size of metastases, in radiography of the lung fields before and after treatment and or on thoracic CT-scan, and or 2) reduced signs and symptoms of pulmonary dysfunction i.e. disappearance of dyspnea, increased FEV1 and VC, eventually combined with an improved SAT O₂ measured using pulse oximetry, provided that the first SAT O₂% was abnormally low.

1. A composition for airway administration to a subject comprising an effective amount of one or more agents capable of inhibiting angiogenesis and/or one or more active anti-cancer agents and/or one or more anti-metastatic agents for use as a medicament for the local inhibition of metastasis in the terminal lung unit (TLU), interalveolar septae, pulmonary interstitium and pulmonary parenchyma.

2. The composition of claim 1, wherein the effective amount of one or more agents capable of inhibiting angiogenesis and/or active anti-cancer agents and/or anti-metastatic agents is administered by intratracheal, intrabronchial, intraalveolar or bronchio-alveolar administration.

3. The composition of claim 1, wherein one or more agents capable of inhibiting angiogenesis and/or active anti-cancer agents and/or anti-metastatic agents is an agent capable of inhibiting VEGF receptor and/or an agent capable of inhibiting any VEGF and/or an agent capable of trapping or inhibiting binding of VEGF to its receptor and/or an agent capable of inhibiting VEGF gene expression on mRNA level.

4. (canceled)

5. The composition of claim 1, wherein one or more agents capable of inhibiting angiogenesis and/or active anti-cancer agents and/or anti-metastatic agents is a small molecule tyrosine kinase inhibitor.

6-7. (canceled)

8. The composition of claim 1, wherein one or more agents capable of inhibiting angiogenesis and/or active anti-cancer agents and/or anti-metastatic agents is thalidomide or a derivative and/or analogue thereof.

9. The composition of claim 1, wherein one or more agents capable of inhibiting angiogenesis and/or active anti-cancer agents and/or anti-metastatic agents is Bevacizumab.

10. The composition of claim 1, wherein one or more agents capable of inhibiting angiogenesis and/or active anti-cancer agents and/or anti-metastatic agents is Suramin.

11. The composition of claim 1, wherein one or more agents capable of inhibiting angiogenesis and/or active anti-cancer agents and/or anti-metastatic agents is Sunitinib.

12. The composition of claim 1, wherein the subject is administered a solution of one or more agents capable of inhibiting angiogenesis and/or active anti-cancer agents and/or anti-metastatic agents via bronchoalveolar lavage and/or via blind tracheal washing.

13. (canceled)

14. The composition of claim 1, wherein the subject is administered a nebulized solution or a suspension of one or more agents capable of inhibiting angiogenesis and/or active anti-cancer agents and/or anti-metastatic agents and/or a nebulized aerosol or inhaled powder form of one or more of said agents.

15. (canceled)

16. The composition of claim 1, wherein the subject is administered a pegylated, liposomal or nanoparticle prepared form of one or more agents capable of inhibiting angiogenesis and/or active anti-cancer agents and/or anti-metastatic agents.

17. The composition of claim 1, wherein the subject is administered one or more agents capable of inhibiting angiogenesis and/or active anti-cancer agents and/or anti-metastatic agents during bronchoscopy.

18. The composition of claim 1, wherein the subject is administered also administrated active anti-cancer agents chemotherapeutics systemically.

19. The composition of claim 1, wherein the subject is a mammal.

20. The composition of claim 1, wherein the mammal is a human.

21. The composition of claim 1, wherein the human is a child younger than 12 years of age.

22. The composition of claim 1, wherein the human is an adult older than 12 years of age.

23-25. (canceled)

26. A composition for airway administration comprising an effective amount of one or more agents capable of inhibiting angiogenesis and/or one or more active anti-cancer agents and/or one or more anti-metastatic agents for use as a medicament for the local inhibition of metastasis in the terminal lung unit (TLU), interalveolar septae, pulmonary interstitium and pulmonary parenchyma.

27. (canceled)

28. A method for the local inhibition of metastasis in the terminal lung unit (TLU), interalveolar septae, pulmonary interstitium and pulmonary parenchyma in a subject comprising airway administration of an effective amount of one or more agents capable of inhibiting angiogenesis and/or one or more active anti-cancer agents and/or one or more anti-metastatic agents.

29. The method according to claim 28, wherein the agent capable of inhibiting angiogenesis is thalidomide or a derivative and/or an analogue thereof.
